(19) United States
(12) Patent Application Publication
(10) Pub. No.: US 2026/0008821 A1
(43) Pub. Date: Jan. 8, 2026

(54) MODIFIED HIV ENVELOPE ANTIGENS AND METHOD OF USE THEREOF

(71) Applicant: THE WISTAR INSTITUTE OF ANATOMY AND BIOLOGY, Philadelphia, PA (US)

(72) Inventors: Daniel Kulp, Moorestown, NJ (US); Rumi Habib, Philadelphia, PA (US)

(21) Appl. No.: 18/994,715

(22) PCT Filed: Jul. 14, 2023
(86) PCT No.: PCT/US2023/070242

§ 371 Date: Jan. 15, 2025

Related U.S. Application Data
(60) Provisional application No. 63/368,507, filed on Jul. 15, 2022.

(51) Int. Cl. C07K 14/005; A61K 39/21; A61P 37/04

(52) U.S. Cl. CPC C07K 14/005; A61K 39/21; A61P 37/04; C12N 2740/16022; C12N 2740/16034

(57) ABSTRACT

Disclosed herein are modified HIV envelope immunogens, engineered nanoparticle vaccines comprising modified HIV envelope immunogens and methods of use thereof for HIV vaccines.

### CROSS-REFERENCE TO RELATED APPLICATIONS

This Application claims priority to, and benefit of, U.S. Provisional Patent Application No. 63/368,507, filed on Jul. 15, 2022, the disclosure of which is hereby incorporated by reference in its entirety.

### BACKGROUND

A central goal of HIV-1 vaccine development is the elicitation of broadly neutralizing antibodies (bNAbs) to the envelope glycoprotein (Env). However, HIV antibodies are generally developed through a complex process of Env-antibody coevolution.

A need remains in the art for the development of HIV antigens that can induce bNAbs against HIV, and for methods of use thereof for or the treatment or prevention of a disease or disorder associated with HIV infection such as AIDS.

### SUMMARY OF THE INVENTION

In one aspect, the invention relates to a nucleic acid molecule encoding a modified HIV envelope immunogen, comprising at least one SHIV-evolution identified mutation. In some embodiments, at least one SHIV-evolution identified mutation is selected from the group consisting of D167N, D167G, N276A, H130N, N187S, Q170R, K171R, R169K, N160D, N160K, and T162A. In some embodiments, the nucleic acid molecule comprises at least two tandem repeats of the immunogen. In some embodiments, the immunogen further comprises at least one selected from the group consisting of: a mutation reverting to WT Env sequence, a CD4-binding site germline targeting mutation, and an Apex N160 glycan modification.

In some embodiments, the immunogen is operably linked to at least one selected from the group consisting of: a leader sequence, an epitope tag, a transmembrane domain, and a nanoparticle self-assembly domain sequence. In some embodiments, the nanoparticle self-assembly domain sequence comprises a Ferritin nanoparticle core comprising a sequence as set forth in SEQ ID NO:72. In some embodiments, the nucleic acid molecule comprises a nucleotide sequence selected from the group consisting of:
(a) the nucleotide sequence encodes a peptide comprising an amino acid sequence having at least about 90% identity over an entire length of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66; and
(b) the nucleotide sequence encodes a peptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66.

In some embodiments, the nucleic acid molecule comprises a nucleotide sequence selected from the group consisting of:
(a) the nucleotide sequence having at least about 90% identity over an entire length of the nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 and SEQ ID NO:65; and
(b) the nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 and SEQ ID NO:65.

In some embodiments, the nucleic acid molecule comprises an expression vector. In some embodiments, the nucleic acid molecule is incorporated into a nanoparticle.

In some aspects, the invention relates to an immunogenic composition comprising at least one nucleic acid molecule of the invention. In some embodiments, the immunogenic composition further comprises a pharmaceutically acceptable excipient. In some embodiments, the immunogenic composition further comprising an adjuvant.

In some aspects, the invention relates to a method of inducing an immune response against HIV in a subject in need thereof, the method comprising administering a nucleic acid molecule of the invention or an immunogenic composition of the invention to the subject. In some embodiments, the method comprises administering includes at least one of electroporation and injection.

In some aspects, the invention relates to a method of protecting a subject in need thereof from infection with HIV, the method comprising administering a nucleic acid molecule of the invention or an immunogenic composition of the invention to the subject.

In some embodiments, the invention relates to a method of treating a subject in need thereof against HIV, the method comprising administering a nucleic acid molecule of the invention or an immunogenic composition of the invention to the subject, wherein the subject is thereby resistant to one or more HIV strains.

In some embodiments, the invention relates to a modified HIV envelope immunogen, comprising at least one SHIV-evolution identified mutation. In some embodiments, at least one SHIV-evolution identified mutation is selected from the group consisting of D167N, D167G, N276A, H130N, N187S, Q170R, K171R, R169K, N160D, N160K, and T162A. In some embodiments, the modified HIV envelope immunogen comprises at least two tandem repeats of the immunogen.

In some embodiments, the immunogen further comprises at least one selected from the group consisting of: a mutation reverting to WT Env sequence, a CD4-binding site germline targeting mutation, and an Apex N160 glycan modification. In some embodiments, the immunogen is operably linked to at least one selected from the group consisting of: a leader sequence, an epitope tag, a transmembrane domain, and a nanoparticle self-assembly domain sequence. In some embodiments, the nanoparticle self-assembly domain sequence comprises a Ferritin nanoparticle core comprising a sequence as set forth in SEQ ID NO:72.

In some embodiments, the modified HIV envelope immunogen comprises a sequence selected from the group consisting of:
(a) an amino acid sequence having at least about 90% identity over an entire length of an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66; and
(b) an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66.

In some embodiments, the modified HIV envelope immunogen comprises the nucleic acid molecule incorporated into a nanoparticle.

In some aspects, the present invention relates to an immunogenic composition comprising a modified HIV envelope immunogen of the invention. In some embodiments, the immunogenic composition further comprises a pharmaceutically acceptable excipient. In some embodiments, the immunogenic composition further comprises an adjuvant.

In some aspects, the invention relates to a method of inducing an immune response against HIV in a subject in need thereof, the method comprising administering a modified HIV envelope immunogen of the invention or an immunogenic composition of the invention comprising a modified HIV envelope immunogen to the subject.

In some aspects, the invention relates to a method of protecting a subject in need thereof from infection with HIV, the method comprising administering a modified HIV envelope immunogen of the invention or an immunogenic composition of the invention comprising a modified HIV envelope immunogen to the subject.

In some aspects, the invention relates to a method of treating a subject in need thereof against HIV, the method comprising administering a modified HIV envelope immunogen of the invention or an immunogenic composition of the invention comprising a modified HIV envelope immunogen to the subject, wherein the subject is thereby resistant to one or more HIV strains.

### BRIEF DESCRIPTION OF THE DRAWINGS

FIG. 1: stability/expression profile of immunogens with engineered mutations recapitulating Env-antibody co-evolution.

FIG. 2: Native-PAGE confirms the identity of the peaks.

FIG. 3: SEC-MALS Show Q23 MD39 and 7S Form A Homogenous Trimer of The Correct Size.

FIG. 4: Q23 trimers have native-like CD4-binding sites.

FIG. 5: Q23 constructs are well-formed trimers.

FIG. 6: constructs with additional stabilizing mutations have less V3 accessibility.

FIG. 7: Q23 trimers have inaccessible CD4-induced epitopes.

FIG. 8: additional antibody binding ELISAs.

FIG. 9: V2 Apex bNAb UCA binding ELISAs.

FIG. 10: negative stain-EM.

FIG. 11: decoration of self-assembling ferritin nanoparticles with Q23 MD39.

FIG. 12: development of immunogens recapitulating progressive viral escape.

FIG. 13: N160-glycan deficient Q23 MD39 can bind V2-apex bNAb UCAs.

### DETAILED DESCRIPTION OF THE INVENTION

The present invention relates to the development of modified HIV envelope immunogens with mutations to recapitulating Env-Antibody coevolution, and methods of use of the modified HIV envelope immunogens for the development of HIV vaccines.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule added to the vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a full-length wild type strain HIV antigen. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a modified HIV envelope immunogens set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants to or is in need of being immunized with the herein described vaccine. The mammal can be a human, chimpanzee, dog, cat, horse, cow, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. MODIFIED ENVELOPE IMMUNOGENS

The invention is based in part on the development of modified HIV envelope immunogens, and nucleic acid molecules encoding the same. In one embodiment, the modified HIV envelope immunogen comprises at least one of a SHIV-evolution identified mutation, a mutation reverting to WT Env sequence, a CD4-binding site germline targeting mutation, an Apex N160 glycan modification, or any combination thereof.

In some embodiments, the modified HIV envelope immunogen comprises at least one SHIV-evolution identified mutation. Exemplary SHIV-evolution identified mutations that can be included in the modified HIV envelope immunogens include, but are not limited to D167N, D167G, N276A, H130N, N187S, Q170R, K171R, R169K, N160D, N160K, T162A, or any combination thereof.

In some embodiments, the modified HIV envelope immunogen comprises at least one reversion of a mutation back to the wild-type sequence. Exemplary reversion mutations include, but are not limited to, H570V and Q363N.

In some embodiments, the modified HIV envelope immunogen comprises at least two, at least three, at least four, at least five, at least six or more than six SHIV-evolution identified mutation. In some embodiments, the modified HIV envelope immunogen comprises at least one, at least two, at least three, at least four, at least five, at least six or more than six SHIV-evolution identified mutation and further comprises a mutation reverting to WT Env sequence, a CD4-binding site germline targeting mutation, an Apex N160 glycan modification, or any combination thereof.

In one embodiment, the nucleotide sequence encoding the modified HIV envelope immunogen further comprises a leader sequence, an epitope tag, a transmembrane domain, a nanoparticle self-assembly domain sequence, or a combination thereof.

In one embodiment, the leader sequence comprises an amino acid sequence as set forth in SEQ ID NO:68 or SEQ ID NO:70. In one embodiment, the leader sequence is encoded by a nucleotide sequence as set forth in SEQ ID NO:67 or SEQ ID NO:69.

In one embodiment, the transmembrane domain comprises a PDGFR transmembrane domain, however the invention should not be seen as limited to the PDGFR transmembrane domain as any domain that can function to anchor the modified HIV immunogen on a cellular membrane surface or particle surface can be used as a transmembrane domain of the invention.

In some embodiments the invention relates to nucleic acid molecules comprise a nucleotide sequence encoding a modified HIV envelope immunogen linked to a nucleotide sequence encoding a nanoparticle scaffold domain. In some embodiments, the nanoparticle scaffold domain comprises a Ferritin nanoparticle core self-assembly domain. In one embodiment, the Ferritin nanoparticle core self-assembly domain comprises an amino acid sequence as set forth in SEQ ID NO:72. In one embodiment, the Ferritin nanoparticle core self-assembly domain is encoded by a nucleotide sequence as set forth in SEQ ID NO:71.

In one embodiment, the nucleotide sequence encoding the modified HIV envelope immunogen comprises at least two tandem repeats of the immunogen sequences.

In one embodiment, the invention relates to a nucleic acid molecule encoding a modified HIV envelope immunogen, a fragment thereof, a variant thereof, or a combination thereof. In some embodiments, the nucleic acid molecule encoding the modified HIV envelope immunogen can comprise a nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 or SEQ ID NO:66, or a fragment or variant thereof.

Immunogenic fragments of SEQ ID NO:2, SEQ ID NO:4, SEQ ID NO: 6, SEQ ID NO:8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO: 58, SEQ ID NO: 60, SEQ ID NO:62, SEQ ID NO:64 or SEQ ID NO:66 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 or SEQ ID NO:66.

In one embodiment, the invention relates to a nucleic acid molecule encoding a modified HIV envelope immunogen, a fragment thereof, a variant thereof, or a combination thereof. In some embodiments, the nucleic acid molecule encoding the modified HIV envelope immunogen can comprise a nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 or SEQ ID NO:65. In some embodiments, the nucleic acid molecule encoding the modified HIV envelope immunogen can comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 or SEQ ID NO:65.

Immunogenic fragments of SEQ ID NO:1, SEQ ID NO:3, SEQ ID NO:5, SEQ ID NO:7, SEQ ID NO:9,SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 or SEQ ID NO:65 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 or SEQ ID NO:65.

### Leader Sequence

In some embodiments, the modified HIV envelope immunogen sequences of the invention are operably linked to at least one leader sequence or a pharmaceutically acceptable salt thereof. In some embodiments, the nucleic acid molecules of the invention encoding the modified HIV envelope immunogen sequences are operably linked to at least one nucleotide sequence encoding a leader sequence or a pharmaceutically acceptable salt thereof. “Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

### Linker Sequence

In some embodiments, the modified HIV envelope immunogen sequences of the invention are operably linked to at least one domain such as a second modified HIV envelope immunogen sequence, a nanoparticle self-assembly sequence, a transmembrane domain, an epitope tag or other domain. In some embodiments, two or more domains are linked using a linker sequence. A linker can be either flexible or rigid or a combination thereof. In one embodiment, the linker is a (GGS) n repeat wherein, the GGS is repeated at least 1, 2, 3, 4, 5, 6, 7, 8, 9, or 10 or more than 10 times.

### Vector

The vaccine can comprise one or more vectors that include a nucleic acid encoding the modified HIV envelope immunogen. The one or more vectors can be capable of expressing the modified HIV envelope immunogen. The vector can have a nucleic acid sequence containing an origin of replication. The vector can be a plasmid, bacteriophage, bacterial artificial chromosome or yeast artificial chromosome. The vector can be either a self-replicating extrachromosomal vector or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

### Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

### Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, or provax, or any other expression vector capable of expressing DNA encoding the antigen and enabling a cell to translate the sequence to an antigen that is recognized by the immune system.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

### Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### Excipients and other Components of the Vaccine

The vaccine may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in an alternative plasmid or are delivered as proteins in combination with the plasmid above in the vaccine. The adjuvant may be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Ser. No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### Nanoparticle Formulations

In one embodiment, the immunogenic composition of the invention may comprise a nanoparticle, including but not limited to a lipid nanoparticle (LNP), comprising modified Env immunogen of the invention, or a LNP comprising a nucleic acid encoding a modified Env immunogen of the invention. In some embodiments, the composition comprises or encodes all or part of a modified Env immunogen of the invention, or an immunogenically functional equivalent thereof. In some embodiments, the composition comprises an mRNA molecule that encodes all or part of a modified Env immunogen of the invention.

In one embodiment, the LNP comprises or encapsulates an RNA molecule encoding at least one amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66, or a fragment or variant thereof.

### Method of Vaccination

Also provided herein is a method of treating, protecting against, and/or preventing disease in a subject in need thereof by administering the vaccine to the subject. Administration of the vaccine to the subject can induce or elicit an immune response in the subject. The induced immune response can be used to treat, prevent, and/or protect against disease, for example, pathologies relating to HIV infection. In one embodiment, the pathology relating to HIV infection is AIDS.

The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies that are reactive to the HIV envelope protein. The induced cellular immune response can include a CD8+ T cell response, which is induced by about 2-fold to about 30-fold, about 3-fold to about 25-fold, or about 4-fold to about 20-fold.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### Administration

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

### Kit

Provided herein is a kit, which can be used for treating a subject using the method of vaccination described above. In one embodiment, the kit can comprise the vaccine. In one embodiment, the kit can comprise a nucleic acid molecule encoding a modified HIV envelope immunogen of the invention.

The kit can also comprise instructions for carrying out the vaccination method described above and/or how to use the kit. Instructions included in the kit can be affixed to packaging material or can be included as a package insert. While instructions are typically written or printed materials, they are not limited to such. Any medium capable of storing instructions and communicating them to an end user is contemplated by this disclosure. Such media include, but are not limited to, links to websites, QR codes, electronic storage media (e.g., magnetic discs, tapes, cartridges), optical media (e.g., CD ROM), and the like. As used herein, the term “instructions” can include the address of an internet site which provides instructions.

The present invention has multiple aspects, illustrated by the following non-limiting examples.

### 3. EXAMPLES

### Example 1: Minimal HIV-1 Env Mutations Guide V2-apex bNAb Development In Vivo and Can Be Recapitulated In Vitro

A central goal of HIV-1 vaccine development is the elicitation of broadly neutralizing antibodies (bNAbs) to the envelope glycoprotein (Env). However, these antibodies develop through a complex process of Env-antibody coevolution. To study this process, we have infected rhesus macaques (RMs) with chimeric simian-human immunodeficiency viruses (SHIVs) bearing primary HIV-1 Envs. By this means we have isolated several novel bNAb lineages targeting the Env V2-apex and identified the patterns of evolution that elicited them.

Single Genome Sequencing (SGS) was used to sequence circulating viruses in SHIV-infected RMs. V2 apex bNAb mAbs were isolated by single-cell sorting of B cells by baiting with heterologous soluble trimer hooks. Soluble recombinant trimers were designed by incorporating stabilizing mutations into the Env sequence, and escape mutations identified from SGS of SHIV infected RMs were incorporated into constructs. Characterization of the constructs was done by ELISA.

Through SGS of circulating plasma viruses in RMs that developed V2-apex bNAbs it was observed that mutations guiding bNAb development are limited to a small section of the Env protein, on the C-strand residues 166-171 and surrounding glycans. Additionally, relatively few (2-4) mutations at these positions are sufficient to guide V2-apex bNAbs to maturity. Incorporation of these mutations into recombinant trimers showed that they recapitulated escape by reducing binding to bNAbs.

These observations show that in RMs infected with diverse virus strains, a limited and convergent set of mutations guides the maturation of V2-apex bNAb lineages. These data corroborate previous observations regarding Env evolution giving rise to V2-apex bNAbs in humans. Furthermore, the data provided demonstrate that these mutations can be recapitulated by recombinant protein immunogens. Overall, these findings have significant implications for vaccine design as they suggest that a few, minimally mutated boosting immunogens may be sufficient for V2-apex bNAb elicitation.

The Experimental results are now described.

Infection of macaques shows that certain HIV-1 strains could make better vaccines than others. Immunogens Recapitulating Env-Antibody Coevolution were developed and several stabilizing strategies were tested as set forth in Table 1:

| Construct | Design Strategy | Citation |
|---|---|---|
| SOSIP | Basic construct | Sanders et al., PLOS Path |
| | | 2013 |
| v5 | Structure guided design | Torrents de la Peña et al., |
| | | Cell Reports 2017 |
| MD39 | Mammalian display of BG505 | Steichen et al., Immunity |
| | SOSIP, mutation to prevent | 2016 |
| | CD4 binding | |
| 7S | v5 + MD39 | Antanasijevicet al., PLOS |
| | | Path 2020 |
| MD64 | Mammalian Display of | Kulpet al., Nature Comms |
| | BG505 MD39, mutation to | 2017 |
| | prevent CD4 binding | |
| RnS-DS | Reversion of rare residues to | Rutten et al., Cell Reports |
| | consensus (“Repair”), | 2018 |
| | Structure guided design | |
| | (“Stabilize”) | |
| olio6 | MD39 + Structure guided | Kulp et al., Nature Comms |
| | design | 2017 |

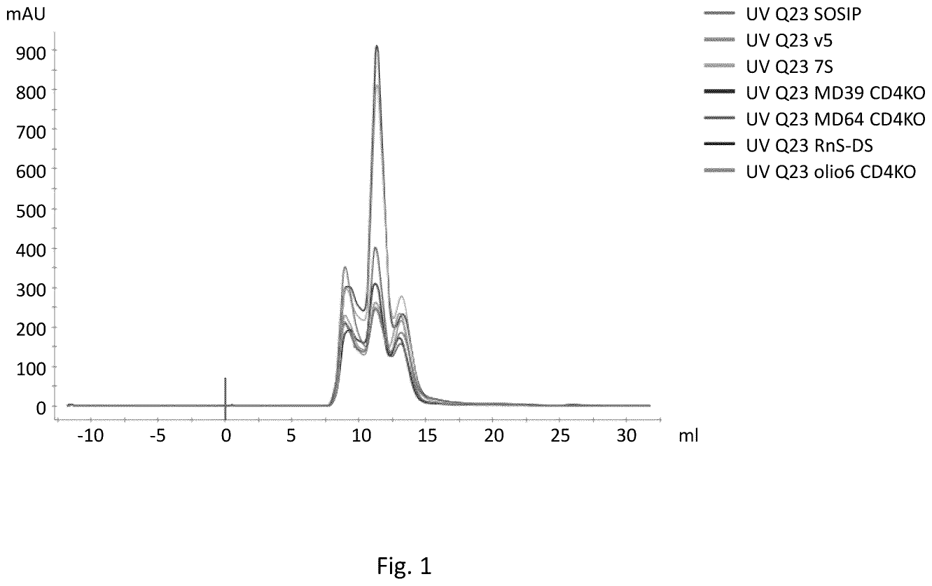

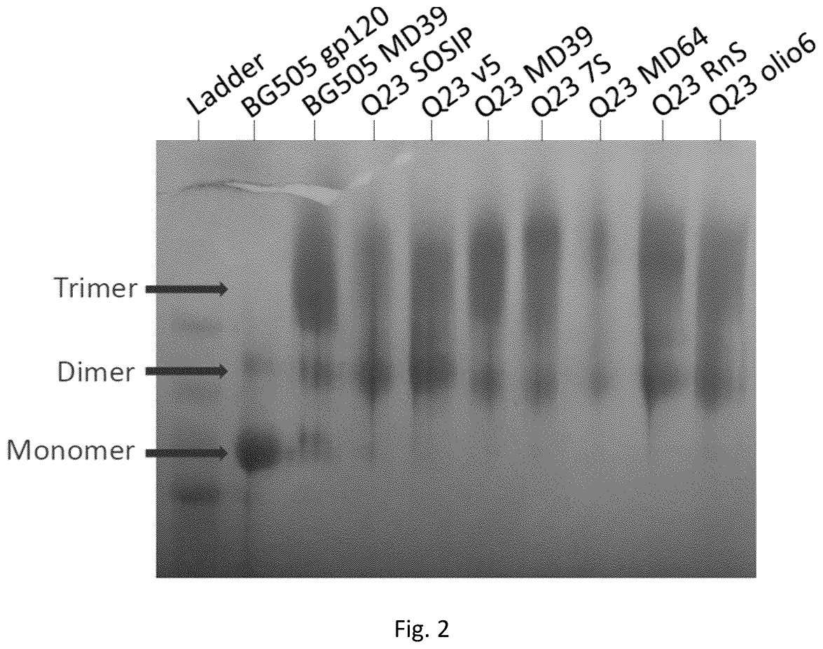

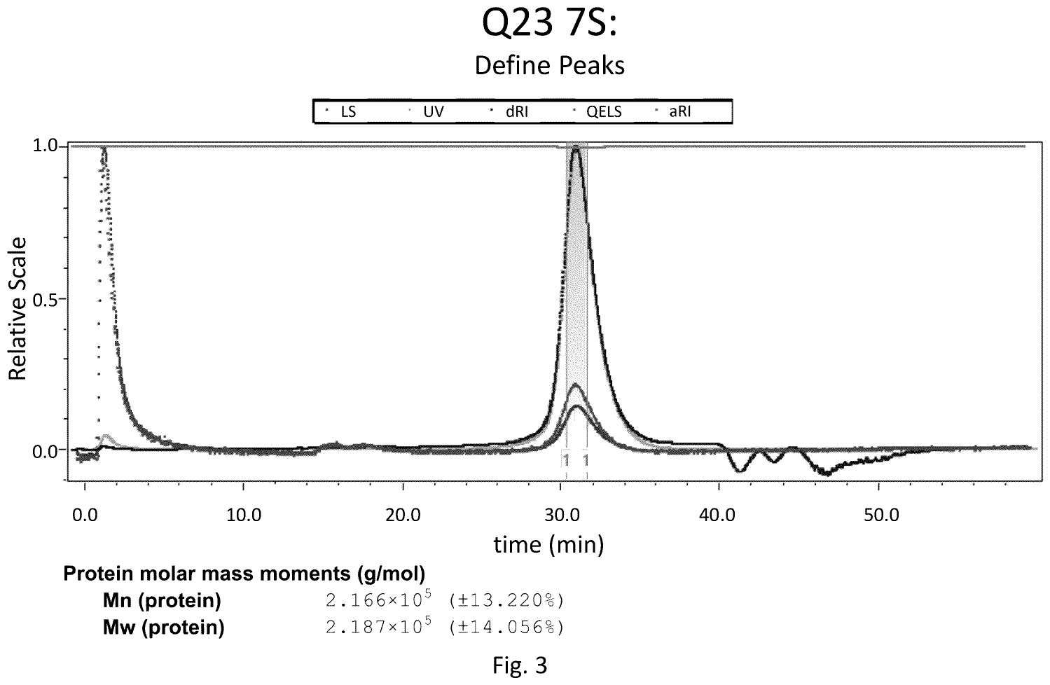

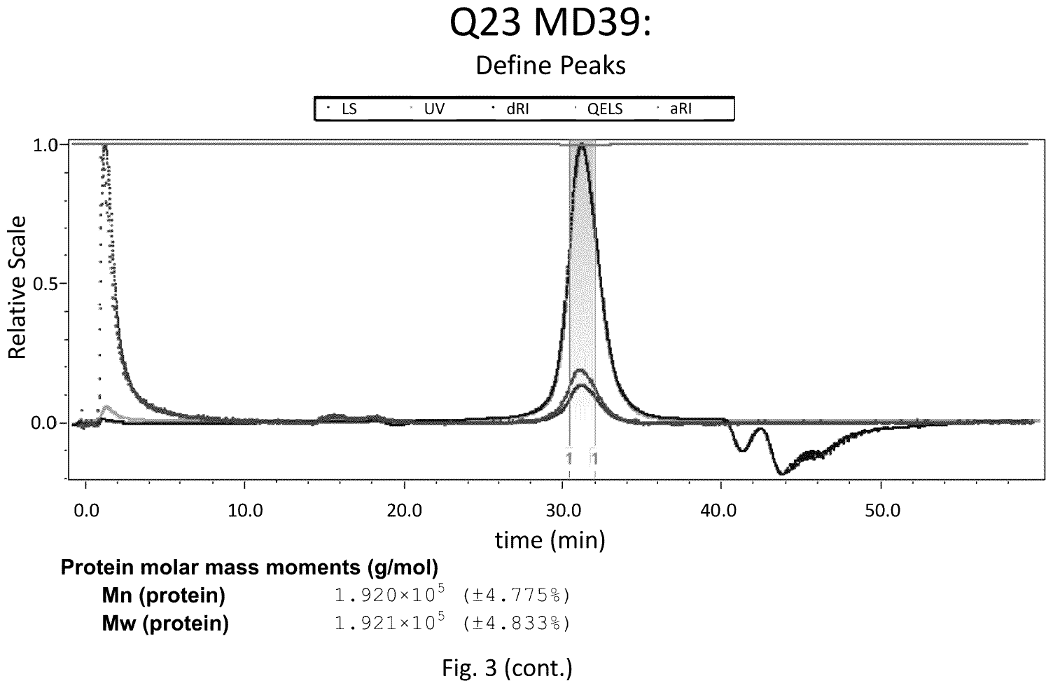

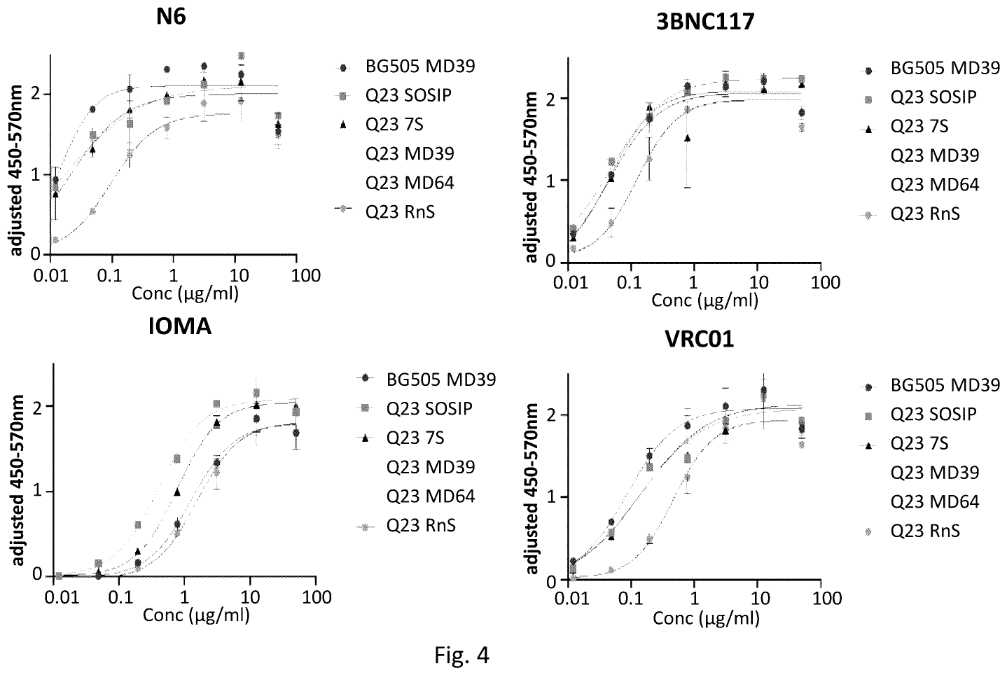

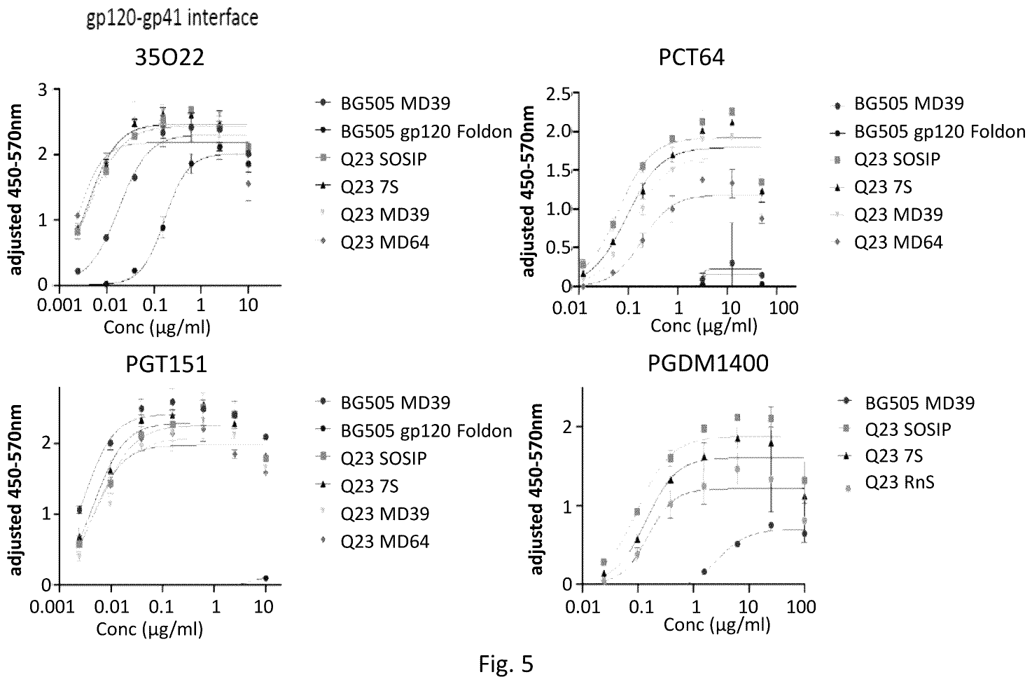

FIG. 1 shows a stability/expression profile of immunogens with engineered mutations recapitulating Env-antibody co-evolution. Q23.MD39 and Q23.7S have the best stability/expression profile. Native-PAGE was used to confirm the identity of the peaks (FIG. 2). Size Exclusion Chromatography-Multiple Angle Light Scattering (SEC-MALS) was used to show that Q23 MD39 and 7S form a homogenous trimer of the correct size (FIG. 3). Q23 trimers are well formed and have native-like CD4-binding sites (FIG. 4 and FIG. 5).

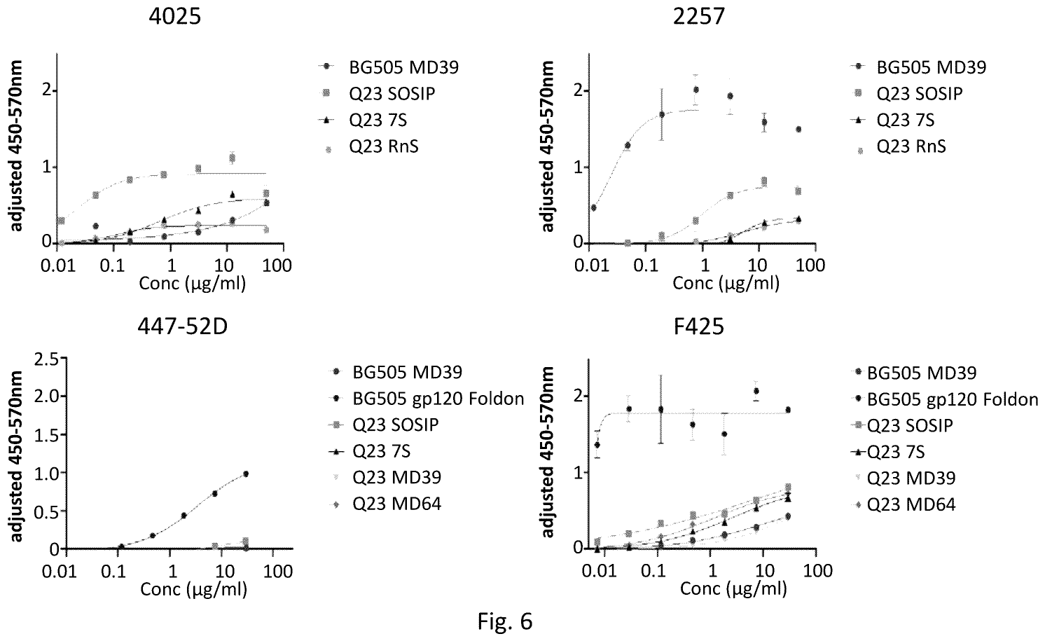

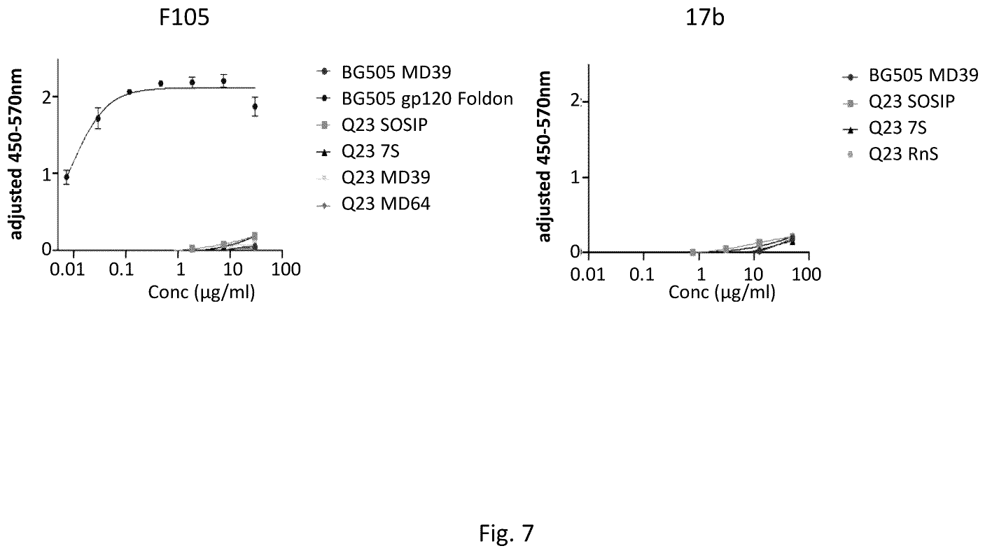

Constructs with additional stabilizing mutations have less V3 accessibility (FIG. 6). Q23 trimers also have inaccessible CD4-induced epitopes (FIG. 7).

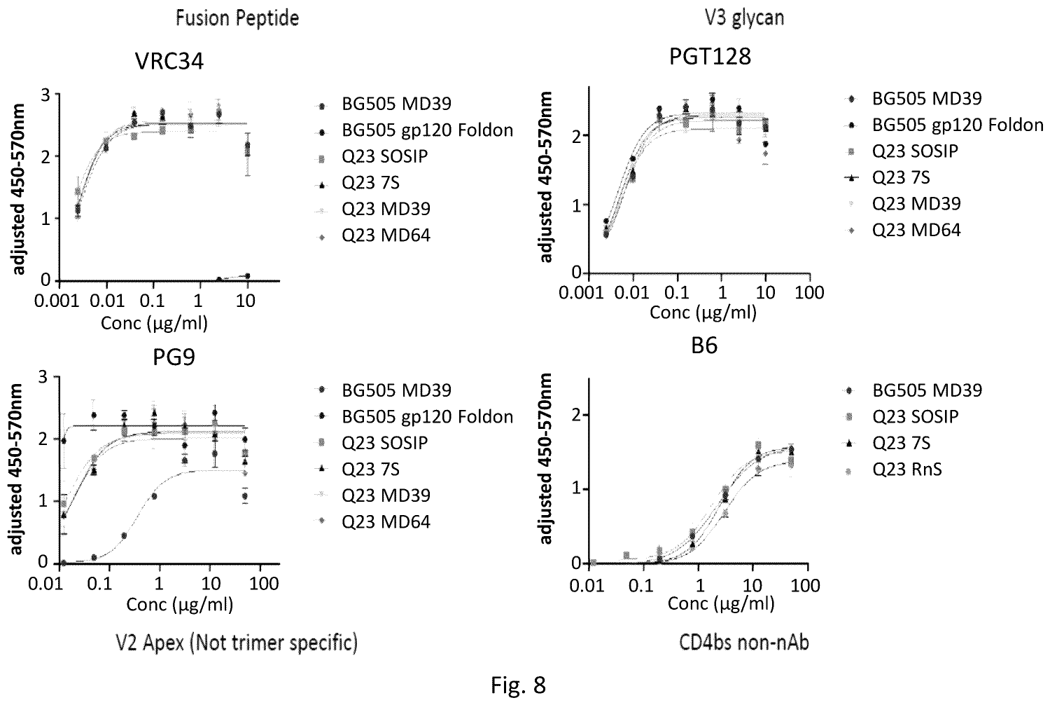

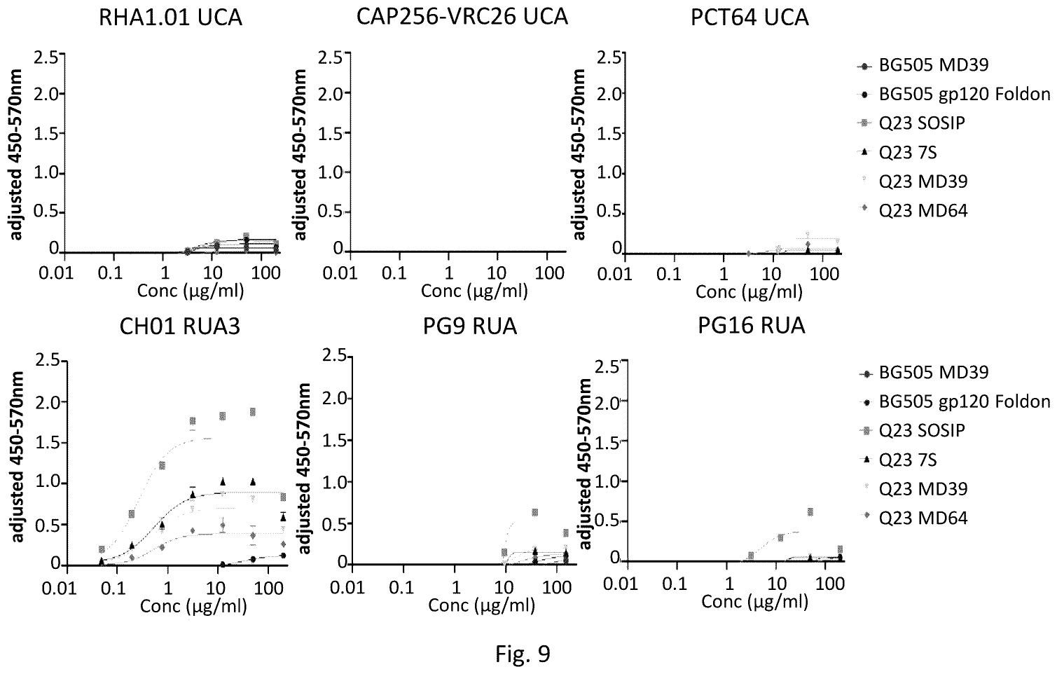

FIG. 8 shows additional antibody binding ELISAs. FIG. 9 shows V2 Apex bNAb UCA binding ELISAs.

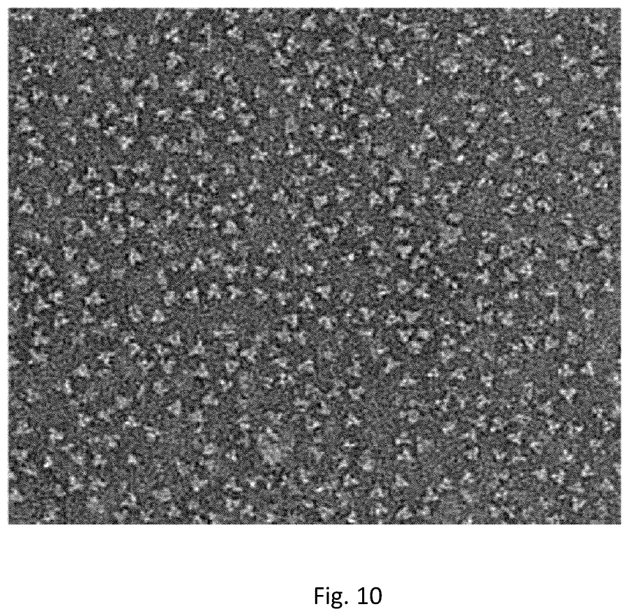

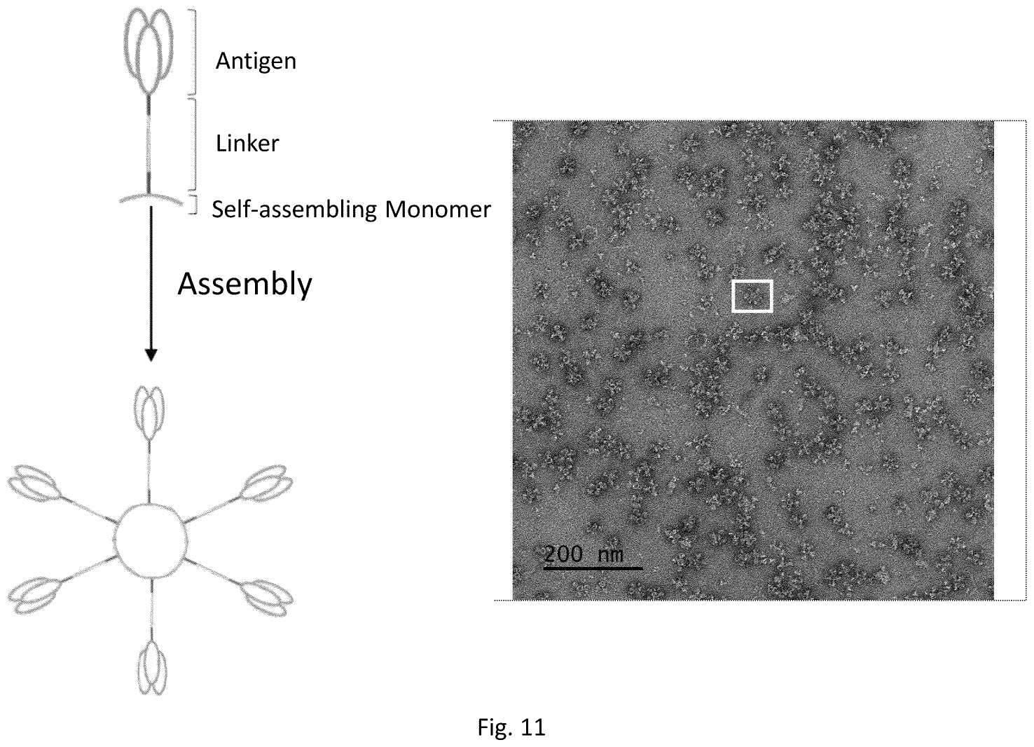

FIG. 10 presents a negative stain-EM. FIG. 11 shows the decoration of self-assembling ferritin nanoparticles with Q23 MD39.

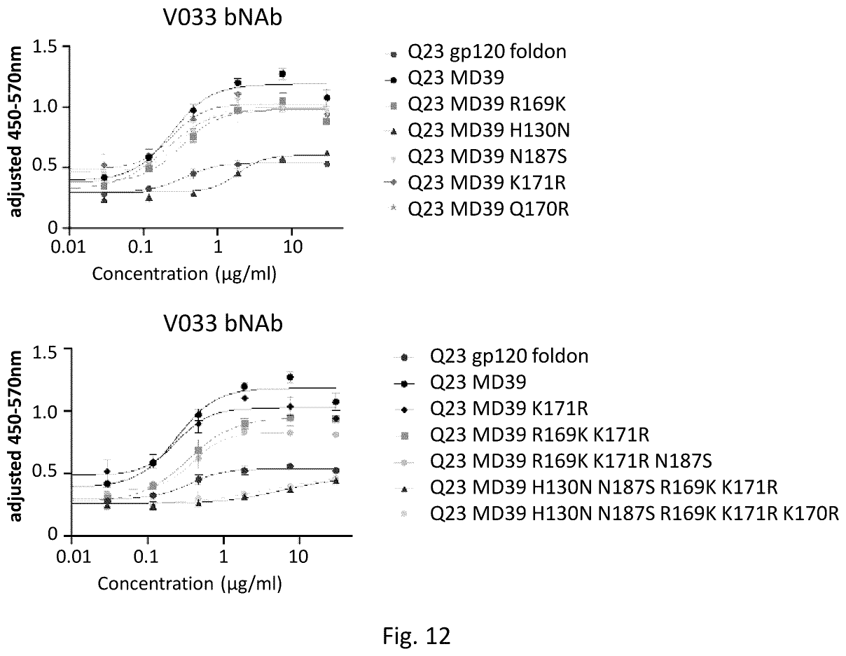

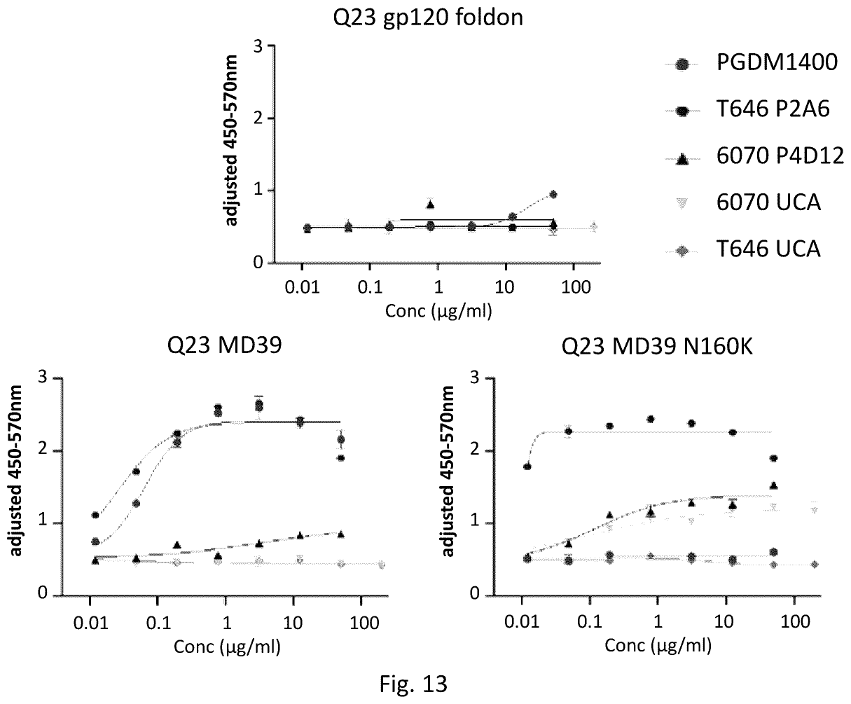

Single genome sequencing of SHIV-infected RMs identifies boosting mutations including 130 (+glycan); G186S (+glycan); V2+glycan; R169K, R172K, H130N (+glycan); Q170R; K171R; N185S (+glycan); D167N/G. FIG. 12 shows the development of immunogens recapitulating progressive viral escape. FIG. 13 provides data demonstrating that N160-glycan deficient Q23 MD39 can bind V2-apex bNAb UCAs.

### Example 2: SEQUENCES

Key:
SHIV-evolution identified mutations
Mutations reverting to WT Env sequence
Glycine/Serine linker domain
PDGFR transmembrane domain
Epitope tag
CD4-binding site germline targeting mutation
Apex N160 glycan modifications
Q23_MD39
SEQ ID NO: 1
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 2-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_Avi
SEQ ID NO: 3-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATGGCCT
GAACGACATCTTCGAGGCGCAGAAAATTGAGTGGCACGAGTGATAA
SEQ ID NO: 4-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELDGLNDIFEAQKIEWHE**
>Q23_MD39_D167N
SEQ ID NO: 5-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGAACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 6-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRNKRQKVYSLFYRLDIVPINENQGSEYRLINCNTSAITQACPKVSFEPIPIHYCT
PAGFAILKCKDEGFNGTGLCKNVSTVQCTHGIKPVVSTQLLLNGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_Ferritin
SEQ ID NO: 7-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATGGCAG
CGGAGGGCTTTCAAAAGATATTATTAAGCTCCTGAATGAGCAAGTCAATAAAGAAATGCAATCTTCT
AACCTGTACATGAGCATGTCTAGCTGGTGTTATACTCACAGTCTCGACGGCGCTGGCCTCTTTCTGT
TCGATCACGCCGCTGAAGAATATGAGCACGCGAAGAAGCTTATAATCTTCCTGAACGAGAATAATGT
TCCCGTCCAACTGACGTCCATTTCCGCCCCTGAACACAAGTTTGAGGGTCTGACTCAGATCTTTCAA
AAGGCGTACGAGCACGAGCAGCATATCAGCGAGAGCATTAACAACATTGTCGATCACGCCATTAAAA
GCAAAGACCACGCTACCTTCAACTTTCTCCAATGGTACGTCGCCGAACAGCATGAGGAAGAAGTATT
GTTCAAGGATATACTCGATAAGATTGAACTGATCGGAAATGAGAATCACGGCCTGTACCTGGCCGAC
CAATACGTCAAAGGCATTGCCAAGTCTAGGAAAAGCTAGTAG
SEQ ID NO: 8-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELDGSGGLSKDIIKLLNEQVNKEMQSS
NLYMSMSSWCYTHSLDGAGLFLFDHAAEEYEHAKKLIIFLNENNVPVQLTSISAPEHKFEGLTQIFQ
KAYEHEQHISESINNIVDHAIKSKDHATFNFLQWYVAEQHEEEVLFKDILDKIELIGNENHGLYLAD
QYVKGIAKSRKS**
>Q23_MD39_Ferritin_N276A_pVax
SEQ ID NO: 9-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGGCTATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATGGCAG
CGGAGGGCTTTCAAAAGATATTATTAAGCTCCTGAATGAGCAAGTCAATAAAGAAATGCAATCTTCT
AACCTGTACATGAGCATGTCTAGCTGGTGTTATACTCACAGTCTCGACGGCGCTGGCCTCTTTCTGT
TCGATCACGCCGCTGAAGAATATGAGCACGCGAAGAAGCTTATAATCTTCCTGAACGAGAATAATGT
TCCCGTCCAACTGACGTCCATTTCCGCCCCTGAACACAAGTTTGAGGGTCTGACTCAGATCTTTCAA
AAGGCGTACGAGCACGAGCAGCATATCAGCGAGAGCATTAACAACATTGTCGATCACGCCATTAAAA
GCAAAGACCACGCTACCTTCAACTTTCTCCAATGGTACGTCGCCGAACAGCATGAGGAAGAAGTATT
GTTCAAGGATATACTCGATAAGATTGAACTGATCGGAAATGAGAATCACGGCCTGTACCTGGCCGAC
CAATACGTCAAAGGCATTGCCAAGTCTAGGAAAAGCTAGTAG
SEQ ID NO: 10-
MDWTWILFLVAAATRVHSAVENLWVTVYYGVPVWRDADTTLFCASDAKAYETEKHNVW
ATHACVPTDPNPQEIHLDNVTEKFNMWKNNMVEQMHEDIISLWDQSLKPCVKLTPLCV
TLHCTNVTSVNTTGDREGLKNCSFNMTTELRDKRQKVYSLFYRLDIVPINENQGSEYR
LINCNTSAITQACPKVSFEPIPIHYCTPAGFAILKCKDEGFNGTGLCKNVSTVQCTHG
IKPVVSTQLLLNGSLAEKNIIIRSEAITNNAKIIIVQLVQPVTIKCIRPNNNTVKSIR
IGPGQAFYYTGDIIGDIRQAHCNVTRSRWNKTLQEVAEKLRTYFGNKTIIFAQSSGGD
LEITTHSFNCGGEFFYCNTSGLFNSTWYVNSTWNDTDSTQESNDTITLPCRIKQIINM
WQRAGQAMYAPPIPGVIKCESNITGLLLTRDGGKDNNVNETFRPGGSDMRDNWRSELY
KYKVVEIEPLGVAPTRCKRRVVERRRRRRAVGIGAVSLGFLGAAGSTMGAASITLTVQ
ARQLLSGIVQQQNNLLRAPEPQQHLLKDTHWGIKQLQARVLAVEHYLRDQQLLGIWGC
SGKLICCTNVPWNSSWSNKSLDEIWNNMTWLQWDKEINNYTQLIYRLIEESQNQQEKN
EKELLELDGSGGLSKDIIKLLNEQVNKEMQSSNLYMSMSSWCYTHSLDGAGLFLFDHA
AEEYEHAKKLIIFLNENNVPVQLTSISAPEHKFEGLTQIFQKAYEHEQHISESINNIV
DHAIKSKDHATFNFLQWYVAEQHEEEVLFKDILDKIELIGNENHGLYLADQYVKGIAK
SRKS
>Q23_MD39_H130N
SEQ ID NO: 11-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGAACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 12
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLNCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_H130N_N187S
SEQ ID NO: 13-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGAACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 14-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLNCTNVTSVNTTGDREG
LKNCSFNMTTELRDKRQKVYSLFYRLDIVPINESQGSEYRLINCNTSAITQACPKVSFEPIPIHYCT
PAGFAILKCKDEGFNGTGLCKNVSTVQCTHGIKPVVSTQLLLNGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_H130N_N187S_Q170R_K171R
SEQ ID NO: 15-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGAACTGTACCAATGTGACATCCGTGAACACCACAGGCGACAGAGAGGGC
CTGAAGAATTGTTCTTTCAACATGACCACAGAGCTGAGGGACAAGCGCCGGAGAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 16-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLNCTNVTSVNTTGDREG
LKNCSFNMTTELRDKRRRVYSLFYRLDIVPINESQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_H130N_N187S_R169K
SEQ ID NO: 17-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGAACTGTACCAATGTGACATCCGTGAACACCACAGGCGACAGAGAGGGC
CTGAAGAATTGTTCTTTCAACATGACCACAGAGCTGAGGGACAAGAAGCAGAAAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 18-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLNCTNVTSVNTTGDREG
LKNCSFNMTTELRDKKQKVYSLFYRLDIVPINESQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_H130N_N187S_R169K_K171R
SEQ ID NO: 19-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGAACTGTACCAATGTGACATCCGTGAACACCACAGGCGACAGAGAGGGC
CTGAAGAATTGTTCTTTCAACATGACCACAGAGCTGAGGGACAAGAAGCAGAGAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 20-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLNCTNVTSVNTTGDREG
LKNCSFNMTTELRDKKQRVYSLFYRLDIVPINESQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_H130N_N187S_R169K_Q170R_K171R
SEQ ID NO: 21-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGAACTGTACCAATGTGACATCCGTGAACACCACAGGCGACAGAGAGGGC
CTGAAGAATTGTTCTTTCAACATGACCACAGAGCTGAGGGACAAGAAGCGGAGAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 22-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLNCTNVTSVNTTGDREG
LKNCSFNMTTELRDKKRRVYSLFYRLDIVPINESQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_H570V
SEQ ID NO: 23-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCGTCTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 24-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLLNGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTVWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_K171R
SEQ ID NO: 25-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAGAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 26-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQRVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_N160D
SEQ ID NO: 27-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCGACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 28-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFDMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLLNGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_N160K
SEQ ID NO: 29-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAAGATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 30-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFKMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_N187S
SEQ ID NO: 31-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 32-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFYRLDIVPINESQGSEYRLINCNTSAITQACPKVSFEPIPIHYCT
PAGFAILKCKDEGFNGTGLCKNVSTVQCTHGIKPVVSTQLLLNGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_N187S_K171R
SEQ ID NO: 33-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAGAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 34-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQRVYSLFYRLDIVPINESQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_N187S_Q170R_K171R
SEQ ID NO: 35-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCGGAGAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 36-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRRRVYSLFYRLDIVPINESQGSEYRLINCNTSAITQACPKVSFEPIPIHYCT
PAGFAILKCKDEGFNGTGLCKNVSTVQCTHGIKPVVSTQLLLNGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_PDGFR
SEQ ID NO: 37-
ATGGAGACAGATACACTTCTGTTATGGGTGCTGTTGCTGTGGGTGCCGGGGTCCACAGGCGACGCCG
TGGAGAACCTGTGGGTGACCGTGTACTATGGCGTGCCCGTGTGGAGGGACGCAGATACCACACTGTT
CTGCGCCTCCGACGCCAAGGCCTACGAGACAGAGAAGCACAACGTGTGGGCAACCCACGCATGCGTG
CCAACAGACCCAAATCCCCAGGAGATCCACCTGGATAATGTGACCGAGAAGTTTAACATGTGGAAGA
ACAATATGGTGGAGCAGATGCACGAGGACATCATCTCTCTGTGGGATCAGAGCCTGAAGCCATGCGT
GAAGCTGACCCCCCTGTGCGTGACACTGCACTGTACCAATGTGACATCCGTGAACACCACAGGCGAC
AGAGAGGGCCTGAAGAATTGTTCTTTCAACATGACCACAGAGCTGAGGGACAAGCGCCAGAAAGTGT
ACAGCCTGTTTTATCGGCTGGACATCGTGCCTATCAATGAGAACCAGGGCTCCGAGTACCGCCTGAT
CAATTGCAACACCTCTGCCATCACACAGGCCTGTCCCAAGGTGAGCTTCGAGCCTATCCCAATCCAC
TATTGCACCCCTGCCGGCTTCGCCATCCTGAAGTGTAAGGATGAGGGCTTTAATGGCACAGGCCTGT
GCAAGAACGTGTCCACCGTGCAGTGTACACACGGCATCAAGCCAGTGGTGTCTACCCAGCTGCTGCT
GAATGGCAGCCTGGCCGAGAAGAACATCATCATCCGGTCTGAGAATATCACAAACAATGCCAAGATC
ATCATCGTGCAGCTGGTGCAGCCTGTGACCATCAAGTGCATCAGACCAAACAATAACACAGTGAAGT
CTATCAGGATCGGACCAGGACAGGCCTTTTACTATACCGGCGACATCATCGGCGACATCAGGCAGGC
CCACTGTAACGTGACCCGGAGCAGATGGAATAAGACACTGCAGGAGGTGGCCGAGAAGCTGCGCACC
TATTTCGGCAACAAGACAATCATCTTTGCCCAGAGCTCCGGCGGCGACCTGGAGATCACCACACACA
GCTTCAACTGCGGCGGCGAGTTCTTTTACTGTAATACCTCCGGCCTGTTTAACTCTACCTGGTATGT
GAACAGCACATGGAACGACACCGATAGCACACAGGAGTCCAACGATACCATCACACTGCCATGCAGG
ATCAAGCAGATCATCAATATGTGGCAGAGGGCAGGACAGGCAATGTACGCACCACCTATCCCCGGCG
TGATCAAGTGTGAGAGCAACATCACCGGACTGCTGCTGACACGGGACGGAGGCAAGGATAATAACGT
GAACGAGACCTTCAGACCAGGAGGATCCGACATGAGGGATAATTGGAGATCCGAGCTGTACAAGTAT
AAGGTGGTGGAGATCGAGCCACTGGGAGTGGCACCTACAAGGTGCAAGAGGAGAGTGGTGGAGAGGC
GCCGGAGAAGGCGCGCAGTGGGCATCGGAGCCGTGTCCCTGGGCTTTCTGGGAGCAGCAGGCAGCAC
CATGGGAGCAGCCTCCATCACCCTGACAGTGCAGGCAAGGCAGCTGCTGTCCGGAATCGTGCAGCAG
CAGAATAACCTGCTGAGAGCACCAGAGCCTCAGCAGCACCTGCTGAAGGACACCCACTGGGGCATCA
AGCAGCTGCAGGCCAGGGTGCTGGCCGTGGAGCACTATCTGAGAGACCAGCAGCTGCTGGGCATCTG
GGGCTGCTCTGGCAAGCTGATCTGCTGTACCAATGTGCCTTGGAACTCTAGCTGGTCCAACAAGTCT
CTGGACGAGATCTGGAATAACATGACCTGGCTGCAGTGGGATAAGGAGATCAATAACTACACACAGC
TGATCTATCGCCTGATCGAGGAGAGCCAGAATCAGCAGGAGAAGAACGAGAAGGAGCTGCTGGAGCT
GGATGGCGGTTCTGGGGGCGCGGGGGGCAGCGAGCAGAAGCTCATAAGTGAAGAGGACTTGGGCGGT
TCAGGAGGAGCAGGTGGGTCCGCAGTAGGACAGGACACCCAGGAGGTAATCGTCGTACCTCATAGTC
TCCCATTCAAAGTTGTCGTTATTTCTGCCATTCTTGCACTGGTGGTGCTCACCATAATCAGCCTTAT
CATCCTGATTATGTTGTGGCAGAAGAAGCCACGTTGATGA
SEQ ID NO: 38-
METDTLLLWVLLLWVPGSTGDAVENLWVTVYYGVPVWRDADTTLFCASDAKAYETEKHNVWATHACV
PTDPNPQEIHLDNVTEKFNMWKNNMVEQMHEDIISLWDQSLKPCVKLTPLCVTLHCTNVTSVNTTGD
REGLKNCSFNMTTELRDKRQKVYSLFYRLDIVPINENQGSEYRLINCNTSAITQACPKVSFEPIPIH
YCTPAGFAILKCKDEGFNGTGLCKNVSTVQCTHGIKPVVSTQLLINGSLAEKNIIIRSENITNNAKI
IIVQLVQPVTIKCIRPNNNTVKSIRIGPGQAFYYTGDIIGDIRQAHCNVTRSRWNKTLQEVAEKLRT
YFGNKTIIFAQSSGGDLEITTHSFNCGGEFFYCNTSGLFNSTWYVNSTWNDTDSTQESNDTITLPCR
IKQIINMWQRAGQAMYAPPIPGVIKCESNITGLLLTRDGGKDNNVNETFRPGGSDMRDNWRSELYKY
KVVEIEPLGVAPTRCKRRVVERRRRRRAVGIGAVSLGFLGAAGSTMGAASITLTVQARQLLSGIVQQ
QNNLLRAPEPQQHLLKDTHWGIKQLQARVLAVEHYLRDQQLLGIWGCSGKLICCTNVPWNSSWSNKS
LDEIWNNMTWLQWDKEINNYTQLIYRLIEESQNQQEKNEKELLELDGGSGGAGGSEQKLISEEDLGG
SGGAGGSAVGQDTQEVIVVPHSLPFKVVVISAILALVVLTIISLIILIMLWQKKPR**
>Q23_MD39_Q170R
SEQ ID NO: 39-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCGGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 40-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRRKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_Q363N
SEQ ID NO: 41-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCAATAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 42-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFANSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_R169K
SEQ ID NO: 43-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGAAGCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 44-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKKQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_R169K_K171R
SEQ ID NO: 45-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGAAGCAGAGAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 46-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKKQRVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_R169K_K171R_N187S
SEQ ID NO: 47-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGAAGCAGAGAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 48-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKKQRVYSLFYRLDIVPINESQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_R169K_N187S
SEQ ID NO: 49-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGAAGCAGAAAGTGTACAGCCTGT
TTTATCGGCTGGACATCGTGCCTATCAATGAGAGCCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 50-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKKQKVYSLFYRLDIVPINESQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD39_SpyTag
SEQ ID NO: 51-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATGGCGG
AAGCGGGTCCGCCCATATCGTGATGGTGGACGCTTATAAACCGACAAAATGATAA
SEQ ID NO: 52-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELDGGSGSAHIVMVDAYKPTK**
>Q23_MD39_T162A
SEQ ID NO: 53-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGGCCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 54-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMATELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_7S
SEQ ID NO: 55-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAAAGAAGCACAACGTGTGGGCAACCCACTGCTGCGTGCCAACAGAC
CCAAATCCCCAGGAGATCCACCTGGATAATGTGACCGAGAAGTTTAACATGTGGAAGAACAATATGG
TGGAGCAGATGCACACAGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGAGGCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCTCTGGGCTTTCTGGGAGCAGCAGGCAGCACCATGGGAGC
AGCCTCCATGACCCTGACAGTGCAGGCAAGGAACCTGCTGTCCGGAATCGTGCAGCAGCAGAATAAC
CTGCTGAGAGCACCAGAGTGCCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 56-
MDWTWILFLVAAATRVHSAVENLWVTVYYGVPVWRDADTTLFCASDAKAYETKKHNVWATHCCVPTD
PNPQEIHLDNVTEKFNMWKNNMVEQMHTDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGGDMRDNWRSELYKYKVV
EIEPLGVAPTRCKRRVVERRRRRRAVGIGAVSLGFLGAAGSTMGAASMTLTVQARNLLSGIVQQQNN
LLRAPECQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_MD64
SEQ ID NO: 57-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGAGTGCGTGCCAACAGAC
CCAAATCCCCAGGAGATCCACCTGGATAATGTGACCGAGAAGTTTAACATGTGGAAGAACAATATGG
TGGAGCAGATGCACACAGACATCATCGAGCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCAGACCAAACAATAACACAGTGAAGTCTATCAGGA
TCGGACCAGGACAGGCCTTTTACTATACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCTGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCTCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGCTGACCGTGTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 58-
MDWTWILFLVAAATRVHSAVENLWVTVYYGVPVWRDADTTLFCASDAKAYETEKHNVWATHECVPTD
PNPQEIHLDNVTEKFNMWKNNMVEQMHTDIIELWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIRPNNNTVKSIRIGPGQAFYYTGDIIGDIRQAHCNVTRSRWNKTLQEVAEKLRTYFG
NKTIIFAQSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKLTVWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_Olio6
SEQ ID NO: 59-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACGAGGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTGGCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCATCATCCGGTCTGAGAATATCACAAACAATGCCAAGATCATCATCGTG
CAGCTGGTGCAGCCTGTGACCATCAAGTGCATCGCCCCAAACAATTTCACAGTGAAGTCTATCAGGA
TCGGACCAGGACAGGCCTTTTACTATATGGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCCAGAGCTCCGGCGGCGACCTGGAGATCACCACACACAGCTTCAACT
GCGGCGGCATGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCTG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGATCCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTCCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGCCTCAGCAGCACCTGCTGAAGGACACCCACTGGGGCATCAAGCAGCTGC
AGGCCAGGGTGCTGGCCGTGGAGCACTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 60-
MDWTWILFLVAAATRVHSAVENLWVTVYYGVPVWRDADTTLFCASDAKAYETEKHNVWATHACVPTD
PNPQEIHLDNVTEKFNMWKNNMVEQMHEDIISLWDQSLKPCVKLTPLCVTLHCTNVTSVNTTGDREG
LKNCSFNMTTELRDKRQKVYSLFWRLDIVPINENQGSEYRLINCNTSAITQACPKVSFEPIPIHYCT
PAGFAILKCKDEGFNGTGLCKNVSTVQCTHGIKPVVSTQLLINGSLAEKNIIIRSENITNNAKIIIV
QLVQPVTIKCIAPNNFTVKSIRIGPGQAFYYMGDIIGDIRQAHCNVTRSRWNKTLQEVAEKLRTYFG
NKTIIFAQSSGGDLEITTHSFNCGGMFFYCNTSGLFNSTWYVNSTWNDTDSTQESNDTITLPCRIKL
IINMWQRAGQAMYAPPIPGVIKCESNITGLLLTRDGGKDNNVNETFRPGGSDMRDNWRSELYKYKVV
EIEPLGVAPTRCKRRVVERRRRRRAVGIGAVSLGFLGAAGSTMGAASITLTVQARQLLSGIVQQQNN
LLRAPEPQQHLLKDTHWGIKQLQARVLAVEHYLRDQQLLGIWGCSGKLICCTNVPWNSSWSNKSLDE
IWNNMTWLQWDKEINNYTQLIYRLIEESQNQQEKNEKELLELD**
>Q23_R&S
SEQ ID NO: 61-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACACAGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCTGCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCACCATCCGGTCTGAGAATATCACAAACAATGCCAAGATCATCATCGTG
CAGCTGGTGCAGCCTGTGACCATCAAGTGCATCAGACCAAACAATAACACAAGGAAGTCTATCAGGA
TCGGACCAGGACAGGCCTTTTACGCAACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCAATAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGTGCATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGAGGCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTTCCTGGGCTTTCTGGGAGCAGCAGGCAGCACCATGGGAGC
AGCCTCCAACACCCTGACAGTGCAGGCAAGGCAGCTGCTGTCCGGAATCGTGCAGCAGCAGAATAAC
CTGCCCAGAGCACCAGAGGCACAGCAGCACCTGCTGCAGCTGACCGTGTGGGGCATCAAGCAGCTGC
AGGCCAGGGTGCTGGCCGTGGAGCGGTATCTGGAGGTGCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGATGCAGTGGGATAAGGAGATCAATAACTACACACAGCTGATCTATC
GCCTGATCGAGGAGAGCCAGTTTCAGCAGGAGATCAACGAGGTGGAGCTGCTGGAGCTGGATTGATA
A
SEQ ID NO: 62
MDWTWILFLVAAATRVHSAVENLWVTVYYGVPVWRDADTTLFCASDAKAYETEKHNVWATHACVPTD
PNPQEIHLDNVTEKFNMWKNNMVEQMHTDIISLWDQSLKPCVKLTPLCVTLHCTNVTSVNTTGDREG
LKNCSFNMTTELRDKRQKVYSLFYRLDIVPINENQGSEYRLINCNTSACTQACPKVSFEPIPIHYCT
PAGFAILKCKDEGFNGTGLCKNVSTVQCTHGIKPVVSTQLLLNGSLAEKNITIRSENITNNAKIIIV
QLVQPVTIKCIRPNNNTRKSIRIGPGQAFYATGDIIGDIRQAHCNVTRSRWNKTLQEVAEKLRTYFG
NKTIIFANSSGGDLEITTHSFNCGGEFFYCNTSGLFNSTWYVNSTWNDTDSTQESNDTITLPCRIKQ
IINMWQRAGQCMYAPPIPGVIKCESNITGLLLTRDGGKDNNVNETFRPGGGDMRDNWRSELYKYKVV
EIEPLGVAPTRCKRRVVERRRRRRAVGIGAVFLGFLGAAGSTMGAASNTLTVQARQLLSGIVQQQNN
LPRAPEAQQHLLQLTVWGIKQLQARVLAVERYLEVQQLLGIWGCSGKLICCTNVPWNSSWSNKSLDE
IWNNMTWMQWDKEINNYTQLIYRLIEESQFQQEINEVELLELD**
>Q23_SOSIP
SEQ ID NO: 63
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAGAGAAGCACAACGTGTGGGCAACCCACGCATGCGTGCCAACAGAC
CCAAATCCCCAGGAGATCCACCTGGATAATGTGACCGAGAAGTTTAACATGTGGAAGAACAATATGG
TGGAGCAGATGCACACAGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCACCATCCGGTCTGAGAATATCACAAACAATGCCAAGATCATCATCGTG
CAGCTGGTGCAGCCTGTGACCATCAAGTGCATCAGACCAAACAATAACACAAGGAAGTCTATCAGGA
TCGGACCAGGACAGGCCTTTTACGCAACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCAATAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGAGGCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTTCCTGGGCTTTCTGGGAGCAGCAGGCAGCACCATGGGAGC
AGCCTCCATCACCCTGACAGTGCAGGCAAGGCAGCTGCTGTCCGGAATCGTGCAGCAGCAGAATAAC
CTGCTGAGAGCACCAGAGGCACAGCAGCACCTGCTGAAGCTGACCGTGTGGGGCATCAAGCAGCTGC
AGGCCAGGGTGCTGGCCGTGGAGCGGTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 64
MDWTWILFLVAAATRVHSAVENLWVTVYYGVPVWRDADTTLFCASDAKAYETEKHNVWATHACVPTD
PNPQEIHLDNVTEKFNMWKNNMVEQMHTDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLINGSLAEKNITIRSENITNNAKIIIV
QLVQPVTIKCIRPNNNTRKSIRIGPGQAFYATGDIIGDIRQAHCNVTRSRWNKTLQEVAEKLRTYFG
NKTIIFANSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGGDMRDNWRSELYKYKVV
EIEPLGVAPTRCKRRVVERRRRRRAVGIGAVFLGFLGAAGSTMGAASITLTVQARQLLSGIVQQQNN
LLRAPEAQQHLLKLTVWGIKQLQARVLAVERYLRDQQLLGIWGCSGKLICCTNVPWNSSWSNKSLDE
IWNNMTWLQWDKEINNYTQLIYRLIEESQNQQEKNEKELLELD**
>Q23_v5
SEQ ID NO: 65-
ATGGACTGGACATGGATTCTGTTCCTGGTCGCTGCCGCTACAAGAGTGCATTCCGCCGTGGAGAACC
TGTGGGTGACCGTGTACTATGGCGTGCCCGTGTGGAGGGACGCAGATACCACACTGTTCTGCGCCTC
CGACGCCAAGGCCTACGAGACAAAGAAGCACAACGTGTGGGCAACCCACTGCTGCGTGCCAACAGAC
CCAAATCCCCAGGAGATCCACCTGGATAATGTGACCGAGAAGTTTAACATGTGGAAGAACAATATGG
TGGAGCAGATGCACACAGACATCATCTCTCTGTGGGATCAGAGCCTGAAGCCATGCGTGAAGCTGAC
CCCCCTGTGCGTGACACTGCACTGTACCAATGTGACATCCGTGAACACCACAGGCGACAGAGAGGGC
CTGAAGAATTGTTCTTTCAACATGACCACAGAGCTGAGGGACAAGCGCCAGAAAGTGTACAGCCTGT
TTTATCGGCTGGACATCGTGCCTATCAATGAGAACCAGGGCTCCGAGTACCGCCTGATCAATTGCAA
CACCTCTGCCATCACACAGGCCTGTCCCAAGGTGAGCTTCGAGCCTATCCCAATCCACTATTGCACC
CCTGCCGGCTTCGCCATCCTGAAGTGTAAGGATGAGGGCTTTAATGGCACAGGCCTGTGCAAGAACG
TGTCCACCGTGCAGTGTACACACGGCATCAAGCCAGTGGTGTCTACCCAGCTGCTGCTGAATGGCAG
CCTGGCCGAGAAGAACATCACCATCCGGTCTGAGAATATCACAAACAATGCCAAGATCATCATCGTG
CAGCTGGTGCAGCCTGTGACCATCAAGTGCATCAGACCAAACAATAACACAAGGAAGTCTATCAGGA
TCGGACCAGGACAGGCCTTTTACGCAACCGGCGACATCATCGGCGACATCAGGCAGGCCCACTGTAA
CGTGACCCGGAGCAGATGGAATAAGACACTGCAGGAGGTGGCCGAGAAGCTGCGCACCTATTTCGGC
AACAAGACAATCATCTTTGCCAATAGCTCCGGCGGCGACCTGGAGATCACCACACACAGCTTCAACT
GCGGCGGCGAGTTCTTTTACTGTAATACCTCCGGCCTGTTTAACTCTACCTGGTATGTGAACAGCAC
ATGGAACGACACCGATAGCACACAGGAGTCCAACGATACCATCACACTGCCATGCAGGATCAAGCAG
ATCATCAATATGTGGCAGAGGGCAGGACAGGCAATGTACGCACCACCTATCCCCGGCGTGATCAAGT
GTGAGAGCAACATCACCGGACTGCTGCTGACACGGGACGGAGGCAAGGATAATAACGTGAACGAGAC
CTTCAGACCAGGAGGAGGCGACATGAGGGATAATTGGAGATCCGAGCTGTACAAGTATAAGGTGGTG
GAGATCGAGCCACTGGGAGTGGCACCTACAAGGTGCAAGAGGAGAGTGGTGGAGAGGCGCCGGAGAA
GGCGCGCAGTGGGCATCGGAGCCGTGTTCCTGGGCTTTCTGGGAGCAGCAGGCAGCACCATGGGAGC
AGCCTCCATGACCCTGACAGTGCAGGCAAGGAACCTGCTGTCCGGAATCGTGCAGCAGCAGAATAAC
CTGCTGAGAGCACCAGAGTGCCAGCAGCACCTGCTGAAGCTGACCGTGTGGGGCATCAAGCAGCTGC
AGGCCAGGGTGCTGGCCGTGGAGCGGTATCTGAGAGACCAGCAGCTGCTGGGCATCTGGGGCTGCTC
TGGCAAGCTGATCTGCTGTACCAATGTGCCTTGGAACTCTAGCTGGTCCAACAAGTCTCTGGACGAG
ATCTGGAATAACATGACCTGGCTGCAGTGGGATAAGGAGATCAATAACTACACACAGCTGATCTATC
GCCTGATCGAGGAGAGCCAGAATCAGCAGGAGAAGAACGAGAAGGAGCTGCTGGAGCTGGATTGATA
A
SEQ ID NO: 66-
MDWTWILFLVAAATRVHSAVENLWVTVYYGVPVWRDADTTLFCASDAKAYETKKHNVWATHCCVPTD
PNPQEIHLDNVTEKFNMWKNNMVEQMHTDIISLWDQSLKPCVKLTPLCVTLHCTNVTSVNTTGDREG
LKNCSFNMTTELRDKRQKVYSLFYRLDIVPINENQGSEYRLINCNTSAITQACPKVSFEPIPIHYCT
PAGFAILKCKDEGFNGTGLCKNVSTVQCTHGIKPVVSTQLLLNGSLAEKNITIRSENITNNAKIIIV
QLVQPVTIKCIRPNNNTRKSIRIGPGQAFYATGDIIGDIRQAHCNVTRSRWNKTLQEVAEKLRTYFG
NKTIIFANSSGGDLEITTHSFNCGGEFFYCNTSGLFNSTWYVNSTWNDTDSTQESNDTITLPCRIKQ
IINMWQRAGQAMYAPPIPGVIKCESNITGLLLTRDGGKDNNVNETFRPGGGDMRDNWRSELYKYKVV
EIEPLGVAPTRCKRRVVERRRRRRAVGIGAVFLGFLGAAGSTMGAASMTLTVQARNLLSGIVQQQNN
LLRAPECQQHLLKLTVWGIKQLQARVLAVERYLRDQQLLGIWGCSGKLICCTNVPWNSSWSNKSLDE
IWNNMTWLQWDKEINNYTQLIYRLIEESQNQQEKNEKELLELD**
Leader/Signal Peptide
SEQ ID NO: 67-
ATGGACTGGACATGGATTCTGTTCCTGGTCGCTGCCGCTACAAGAGTGCATTCC
SEQ ID NO: 68-
MDWTWILFLVAAATRVHS
SEQ ID NO: 69-
ATGGAGACAGATACACTTCTGTTATGGGTGCTGTTGCTGTGGGTGCCGGGGTCCACAGGCGAC
SEQ ID NO: 70-
METDTLLLWVLLLWVPGSTGD
Ferritin nanoparticle core:
SEQ ID NO: 71-
CTTTCAAAAGATATTATTAAGCTCCTGAATGAGCAAGTCAATAAAGAAATGCAATCTTCTAACCTGT
ACATGAGCATGTCTAGCTGGTGTTATACTCACAGTCTCGACGGCGCTGGCCTCTTTCTGTTCGATCA
CGCCGCTGAAGAATATGAGCACGCGAAGAAGCTTATAATCTTCCTGAACGAGAATAATGTTCCCGTC
CAACTGACGTCCATTTCCGCCCCTGAACACAAGTTTGAGGGTCTGACTCAGATCTTTCAAAAGGCGT
ACGAGCACGAGCAGCATATCAGCGAGAGCATTAACAACATTGTCGATCACGCCATTAAAAGCAAAGA
CCACGCTACCTTCAACTTTCTCCAATGGTACGTCGCCGAACAGCATGAGGAAGAAGTATTGTTCAAG
GATATACTCGATAAGATTGAACTGATCGGAAATGAGAATCACGGCCTGTACCTGGCCGACCAATACG
TCAAAGGCATTGCCAAGTCTAGGAAAAGC
SEQ ID NO: 72-
LSKDIIKLLNEQVNKEMQSSNLYMSMSSWCYTHSLDGAGLFLFDHAAEEYEHAKKLIIFLNENNVPV
QLTSISAPEHKFEGLTQIFQKAYEHEQHISESINNIVDHAIKSKDHATFNFLQWYVAEQHEEEVLFK
DILDKIELIGNENHGLYLADQYVKGIAKSRKS

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A nucleic acid molecule encoding a modified HIV envelope immunogen, comprising at least one SHIV-evolution identified mutation.

2. The nucleic acid molecule of claim 1, wherein at least one SHIV-evolution identified mutation is selected from the group consisting of D167N, D167G, N276A, H130N, N187S, Q170R, K171R, R169K, N160D, N160K, and T162A.

3. The nucleic acid molecule of claim 1 or claim 2, comprising at least two tandem repeats of the immunogen.

4. The nucleic acid molecule of any one of claims 1-3, wherein the immunogen further comprises at least one selected from the group consisting of: a mutation reverting to WT Env sequence, a CD4-binding site germline targeting mutation, and an Apex N160 glycan modification.

5. The nucleic acid molecule of any one of claims 1-4, wherein the immunogen is operably linked to at least one selected from the group consisting of: a leader sequence, an epitope tag, a transmembrane domain, and a nanoparticle self-assembly domain sequence.

6. The nucleic acid molecule of claim 5, wherein the nanoparticle self-assembly domain sequence comprises a Ferritin nanoparticle core comprising a sequence as set forth in SEQ ID NO:72.

7. The nucleic acid molecule of any one of claims 1-6, wherein the nucleic acid molecule comprises a nucleotide sequence selected from the group consisting of:
(c) the nucleotide sequence encodes a peptide comprising an amino acid sequence having at least about 90% identity over an entire length of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66; and
(d) the nucleotide sequence encodes a peptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66.

8. The nucleic acid molecule of any one of claims 1-7, wherein the nucleic acid molecule comprises a nucleotide sequence selected from the group consisting of:
(e) the nucleotide sequence having at least about 90% identity over an entire length of the nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 and SEQ ID NO:65; and
(f) the nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO: 13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 and SEQ ID NO:65.

9. The nucleic acid molecule of any one of claims 1-8, wherein the nucleic acid molecule comprises an expression vector.

10. The nucleic acid molecule of any one of claims 1-9, wherein the nucleic acid molecule is incorporated into a nanoparticle.

11. An immunogenic composition comprising at least one nucleic acid molecule of any one of claims 1 to 10.

12. The immunogenic composition of claim 11, further comprising a pharmaceutically acceptable excipient.

13. The immunogenic composition of claim 12, further comprising an adjuvant.

14. A method of inducing an immune response against HIV in a subject in need thereof, the method comprising administering a nucleic acid molecule of any one of claims 1 to 10 or an immunogenic composition of any one of claims 11 to 13 to the subject.

15. The method of claim 14, wherein administering includes at least one of electroporation and injection.

16. A method of protecting a subject in need thereof from infection with HIV, the method comprising administering a nucleic acid molecule of any one of claims 1 to 10 or an immunogenic composition of any one of claims 11 to 13 to the subject.

17. A method of treating a subject in need thereof against HIV, the method comprising administering a nucleic acid molecule of any one of claims 1 to 10 or an immunogenic composition of any one of claims 11 to 13 to the subject, wherein the subject is thereby resistant to one or more HIV strains.

18. A modified HIV envelope immunogen, comprising at least one SHIV-evolution identified mutation.

19. The modified HIV envelope immunogen of claim 18, wherein at least one SHIV-evolution identified mutation is selected from the group consisting of D167N, D167G, N276A, H130N, N187S, Q170R, K171R, R169K, N160D, N160K, and T162A.

20. The modified HIV envelope immunogen of claim 18 or claim 19, comprising at least two tandem repeats of the immunogen.

21. The modified HIV envelope immunogen of any one of claims 18-20, wherein the immunogen further comprises at least one selected from the group consisting of: a mutation reverting to WT Env sequence, a CD4-binding site germline targeting mutation, and an Apex N160 glycan modification.

22. The modified HIV envelope immunogen of any one of claims 18-21, wherein the immunogen is operably linked to at least one selected from the group consisting of: a leader sequence, an epitope tag, a transmembrane domain, and a nanoparticle self-assembly domain sequence.

23. The modified HIV envelope immunogen of claim 22, wherein the nanoparticle self-assembly domain sequence comprises a Ferritin nanoparticle core comprising a sequence as set forth in SEQ ID NO:72.

24. The modified HIV envelope immunogen of any one of claims 18-23, wherein the immunogen comprises a sequence selected from the group consisting of:
(g) an amino acid sequence having at least about 90% identity over an entire length of an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66; and
(h) an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64 and SEQ ID NO:66.

25. The modified HIV envelope immunogen of any one of claims 18-24, wherein the nucleic acid molecule is incorporated into a nanoparticle.

26. An immunogenic composition comprising a modified HIV envelope immunogen of any one of claims 18-25.

27. The immunogenic composition of claim 26, further comprising a pharmaceutically acceptable excipient.

28. The immunogenic composition of claim 27, further comprising an adjuvant.

29. A method of inducing an immune response against HIV in a subject in need thereof, the method comprising administering a modified HIV envelope immunogen of any one of claims 18-25 or an immunogenic composition of any one of claims 26-28 to the subject.

30. A method of protecting a subject in need thereof from infection with HIV, the method comprising administering a modified HIV envelope immunogen of any one of claims 18-25 or an immunogenic composition of any one of claims 26-28 to the subject.

31. A method of treating a subject in need thereof against HIV, the method comprising administering a modified HIV envelope immunogen of any one of claims 18-25 or an immunogenic composition of any one of claims 26-28, wherein the subject is thereby resistant to one or more HIV strains.


SEQUENCE LISTING (72 sequences)

SEQ ID NO: 1 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 2 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 3 - DNA, 1989 bp, synthetic construct
  source 1..1989: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggatggcctgaacgacatcttcgaggcgcagaaaattgagtggcacgagtgataa

SEQ ID NO: 4 - protein, 661 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELDGLNDIFEAQKIEWHE

SEQ ID NO: 5 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgaggaacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 6 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRNKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 7 - DNA, 2454 bp, synthetic construct
  source 1..2454: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggatggcagcggagggctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 8 - protein, 816 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELDGSGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 9 - DNA, 2454 bp, synthetic construct
  source 1..2454: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgaggctatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggatggcagcggagggctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 10 - protein, 816 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSEAITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELDGSGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 11 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgaactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 12 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLNCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 13 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgaactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 14 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLNCTNVTSVNTTGDREGLKNCSFNMTTELRDKRQKVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 15 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgaactgtaccaatgtgacatccgtgaacaccacaggcgacagagagggcctgaagaattgttctttcaacatgaccacagagctgagggacaagcgccggagagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 16 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLNCTNVTSVNTTGDREGLKNCSFNMTTELRDKRRRVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 17 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgaactgtaccaatgtgacatccgtgaacaccacaggcgacagagagggcctgaagaattgttctttcaacatgaccacagagctgagggacaagaagcagaaagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 18 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLNCTNVTSVNTTGDREGLKNCSFNMTTELRDKKQKVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 19 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgaactgtaccaatgtgacatccgtgaacaccacaggcgacagagagggcctgaagaattgttctttcaacatgaccacagagctgagggacaagaagcagagagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 20 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLNCTNVTSVNTTGDREGLKNCSFNMTTELRDKKQRVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 21 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgaactgtaccaatgtgacatccgtgaacaccacaggcgacagagagggcctgaagaattgttctttcaacatgaccacagagctgagggacaagaagcggagagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 22 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLNCTNVTSVNTTGDREGLKNCSFNMTTELRDKKRRVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 23 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacaccgtctggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 24 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTVWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 25 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagagagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 26 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQRVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 27 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcgacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 28 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFDMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 29 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaagatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 30 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFKMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 31 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 32 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 33 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagagagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 34 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQRVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 35 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccggagagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 36 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRRRVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 37 - DNA, 2184 bp, synthetic construct
  source 1..2184: mol_type = other DNA
atggagacagatacacttctgttatgggtgctgttgctgtgggtgccggggtccacaggcgacgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggatggcggttctgggggcgcggggggcagcgagcagaagctcataagtgaagaggacttgggcggttcaggaggagcaggtgggtccgcagtaggacaggacacccaggaggtaatcgtcgtacctcatagtctcccattcaaagttgtcgttatttctgccattcttgcactggtggtgctcaccataatcagccttatcatcctgattatgttgtggcagaagaagccacgttgatga

SEQ ID NO: 38 - protein, 726 aa, synthetic construct
METDTLLLWVLLLWVPGSTGDAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELDGGSGGAGGSEQKLISEEDLGGSGGAGGSAVGQDTQEVIVVPHSLPFKVVVISAILALVVLTIISLIILIMLWQKKPR

SEQ ID NO: 39 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccggaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 40 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRRKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 41 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgccaatagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 42 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFANSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 43 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagaagcagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 44 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKKQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 45 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagaagcagagagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 46 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKKQRVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 47 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagaagcagagagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 48 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKKQRVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 49 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagaagcagaaagtgtacagcctgttttatcggctggacatcgtgcctatcaatgagagccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 50 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKKQKVYSLFYRLDIVPINESQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 51 - DNA, 1998 bp, synthetic construct
  source 1..1998: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggatggcggaagcgggtccgcccatatcgtgatggtggacgcttataaaccgacaaaatgataa

SEQ ID NO: 52 - protein, 664 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELDGGSGSAHIVMVDAYKPTK

SEQ ID NO: 53 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatggccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 54 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMATELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 55 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacaaagaagcacaacgtgtgggcaacccactgctgcgtgccaacagacccaaatccccaggagatccacctggataatgtgaccgagaagtttaacatgtggaagaacaatatggtggagcagatgcacacagacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggaggcgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtctctgggctttctgggagcagcaggcagcaccatgggagcagcctccatgaccctgacagtgcaggcaaggaacctgctgtccggaatcgtgcagcagcagaataacctgctgagagcaccagagtgccagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 56 - protein, 646 aa, synthetic construct
MDWTWILFLVAAATRVHSAVENLWVTVYYGVPVWRDADTTLFCASDAKAYETKKHNVWATHCCVPTDPNPQEIHLDNVTEKFNMWKNNMVEQMHTDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGGDMRDNWRSELYKYKVVEIEPLGVAPTRCKRRVVERRRRRRAVGIGAVSLGFLGAAGSTMGAASMTLTVQARNLLSGIVQQQNNLLRAPECQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 57 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgagtgcgtgccaacagacccaaatccccaggagatccacctggataatgtgaccgagaagtttaacatgtggaagaacaatatggtggagcagatgcacacagacatcatcgagctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcagaccaaacaataacacagtgaagtctatcaggatcggaccaggacaggccttttactataccggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatctgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtctctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaagctgaccgtgtggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 58 - protein, 646 aa, synthetic construct
MDWTWILFLVAAATRVHSAVENLWVTVYYGVPVWRDADTTLFCASDAKAYETEKHNVWATHECVPTDPNPQEIHLDNVTEKFNMWKNNMVEQMHTDIIELWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIRPNNNTVKSIRIGPGQAFYYTGDIIGDIRQAHCNVTRSRWNKTLQEVAEKLRTYFGNKTIIFAQSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKLTVWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 59 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacgaggacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttggcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcatcatccggtctgagaatatcacaaacaatgccaagatcatcatcgtgcagctggtgcagcctgtgaccatcaagtgcatcgccccaaacaatttcacagtgaagtctatcaggatcggaccaggacaggccttttactatatgggcgacatcatcggcgacatcaggcaggcccactgtaacgtgacccggagcagatggaataagacactgcaggaggtggccgagaagctgcgcacctatttcggcaacaagacaatcatctttgcccagagctccggcggcgacctggagatcaccacacacagcttcaactgcggcggcatgttcttttactgtaatacctccggcctgtttaactctacctggtatgtgaacagcacatggaacgacaccgatagcacacaggagtccaacgataccatcacactgccatgcaggatcaagctgatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggatccgacatgagggataattggagatccgagctgtacaagtataaggtggtggagatcgagccactgggagtggcacctacaaggtgcaagaggagagtggtggagaggcgccggagaaggcgcgcagtgggcatcggagccgtgtccctgggctttctgggagcagcaggcagcaccatgggagcagcctccatcaccctgacagtgcaggcaaggcagctgctgtccggaatcgtgcagcagcagaataacctgctgagagcaccagagcctcagcagcacctgctgaaggacacccactggggcatcaagcagctgcaggccagggtgctggccgtggagcactatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 60 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHEDIISLWDQSLKPCVKLTPLCVTLHCTNVTSVNTTGDREGLKNCSFNMTTELRDKRQKVYSLFWRLDIVPINENQGSEYRLINCNTSAITQACPKVSFEPIPIHYCTPAGFAILKCKDEGFNGTGLCKNVSTVQCTHGIKPVVSTQLLLNGSLAEKNIIIRSENITNNAKIIIVQLVQPVTIKCIAPNNFTVKSIRIGPGQAFYYMGDIIGDIRQAHCNVTRSRWNKTLQEVAEKLRTYFGNKTIIFAQSSGGDLEITTHSFNCGGMFFYCNTSGLFNSTWYVNSTWNDTDSTQESNDTITLPCRIKLIINMWQRAGQAMYAPPIPGVIKCESNITGLLLTRDGGKDNNVNETFRPGGSDMRDNWRSELYKYKVVEIEPLGVAPTRCKRRVVERRRRRRAVGIGAVSLGFLGAAGSTMGAASITLTVQARQLLSGIVQQQNNLLRAPEPQQHLLKDTHWGIKQLQARVLAVEHYLRDQQLLGIWGCSGKLICCTNVPWNSSWSNKSLDEIWNNMTWLQWDKEINNYTQLIYRLIEESQNQQEKNEKELLELD

SEQ ID NO: 61 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacacagacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgcctgcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcaccatccggtctgagaatatcacaaacaatgccaagatcatcatcgtgcagctggtgcagcctgtgaccatcaagtgcatcagaccaaacaataacacaaggaagtctatcaggatcggaccaggacaggccttttacgcaaccggcgacatcatcggcgacatcaggcaggcccactgtaacgtgacccggagcagatggaataagacactgcaggaggtggccgagaagctgcgcacctatttcggcaacaagacaatcatctttgccaatagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacagtgcatgtacgcaccacctatccccggcgtgatcaagtgtgagagcaacatcaccggactgctgctgacacgggacggaggcaaggataataacgtgaacgagaccttcagaccaggaggaggcgacatgagggataattggagatccgagctgtacaagtataaggtggtggagatcgagccactgggagtggcacctacaaggtgcaagaggagagtggtggagaggcgccggagaaggcgcgcagtgggcatcggagccgtgttcctgggctttctgggagcagcaggcagcaccatgggagcagcctccaacaccctgacagtgcaggcaaggcagctgctgtccggaatcgtgcagcagcagaataacctgcccagagcaccagaggcacagcagcacctgctgcagctgaccgtgtggggcatcaagcagctgcaggccagggtgctggccgtggagcggtatctggaggtgcagcagctgctgggcatctggggctgctctggcaagctgatctgctgtaccaatgtgccttggaactctagctggtccaacaagtctctggacgagatctggaataacatgacctggatgcagtgggataaggagatcaataactacacacagctgatctatcgcctgatcgaggagagccagtttcagcaggagatcaacgaggtggagctgctggagctggattgataa

SEQ ID NO: 62 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHTDIISLWDQSLKPCVKLTPLCVTLHCTNVTSVNTTGDREGLKNCSFNMTTELRDKRQKVYSLFYRLDIVPINENQGSEYRLINCNTSACTQACPKVSFEPIPIHYCTPAGFAILKCKDEGFNGTGLCKNVSTVQCTHGIKPVVSTQLLLNGSLAEKNITIRSENITNNAKIIIVQLVQPVTIKCIRPNNNTRKSIRIGPGQAFYATGDIIGDIRQAHCNVTRSRWNKTLQEVAEKLRTYFGNKTIIFANSSGGDLEITTHSFNCGGEFFYCNTSGLFNSTWYVNSTWNDTDSTQESNDTITLPCRIKQIINMWQRAGQCMYAPPIPGVIKCESNITGLLLTRDGGKDNNVNETFRPGGGDMRDNWRSELYKYKVVEIEPLGVAPTRCKRRVVERRRRRRAVGIGAVFLGFLGAAGSTMGAASNTLTVQARQLLSGIVQQQNNLPRAPEAQQHLLQLTVWGIKQLQARVLAVERYLEVQQLLGIWGCSGKLICCTNVPWNSSWSNKSLDEIWNNMTWMQWDKEINNYTQLIYRLIEESQFQQEINEVELLELD

SEQ ID NO: 63 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacagagaagcacaacgtgtgggcaacccacgcatgcgtgccaacagacccaaatccccaggagatccacctggataatgtgaccgagaagtttaacatgtggaagaacaatatggtggagcagatgcacacagacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcaccatccggtctgagaatatcacaaacaatgccaagatcatcatcgtgcagctggtgcagcctgtgaccatcaagtgcatcagaccaaacaataacacaaggaagtctatcaggatcggaccaggacaggccttttacgcaaccggcgacatcatcggcgacatcaggcaggcccactgtaacgtgacccggagcagatggaataagacactgcaggaggtggccgagaagctgcgcacctatttcggcaacaagacaatcatctttgccaatagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggaggcgacatgagggataattggagatccgagctgtacaagtataaggtggtggagatcgagccactgggagtggcacctacaaggtgcaagaggagagtggtggagaggcgccggagaaggcgcgcagtgggcatcggagccgtgttcctgggctttctgggagcagcaggcagcaccatgggagcagcctccatcaccctgacagtgcaggcaaggcagctgctgtccggaatcgtgcagcagcagaataacctgctgagagcaccagaggcacagcagcacctgctgaagctgaccgtgtggggcatcaagcagctgcaggccagggtgctggccgtggagcggtatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 64 - protein, 646 aa, synthetic construct
MDWTWILFLVAAATRVHSAVENLWVTVYYGVPVWRDADTTLFCASDAKAYETEKHNVWATHACVPTDPNPQEIHLDNVTEKFNMWKNNMVEQMHTDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNITIRSENITNNAKIIIVQLVQPVTIKCIRPNNNTRKSIRIGPGQAFYATGDIIGDIRQAHCNVTRSRWNKTLQEVAEKLRTYFGNKTIIFANSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGGDMRDNWRSELYKYKVVEIEPLGVAPTRCKRRVVERRRRRRAVGIGAVFLGFLGAAGSTMGAASITLTVQARQLLSGIVQQQNNLLRAPEAQQHLLKLTVWGIKQLQARVLAVERYLRDQQLLGIWGCSGKLICCTNVPWNSSWSNKSLDEIWNNMTWLQWDKEINNYTQLIYRLIEESQNQQEKNEKELLELD

SEQ ID NO: 65 - DNA, 1944 bp, synthetic construct
  source 1..1944: mol_type = other DNA
atggactggacatggattctgttcctggtcgctgccgctacaagagtgcattccgccgtggagaacctgtgggtgaccgtgtactatggcgtgcccgtgtggagggacgcagataccacactgttctgcgcctccgacgccaaggcctacgagacaaagaagcacaacgtgtgggcaacccactgctgcgtgccaacagacccaaatccccaggagatccacctggataatgtgaccgagaagtttaacatgtggaagaacaatatggtggagcagatgcacacagacatcatctctctgtgggatcagagcctgaagccatgcgtgaagctgacccccctgtgcgtgacactgcactgtaccaatgtgacatccgtgaacaccacaggcgacagagagggcctgaagaattgttctttcaacatgaccacagagctgagggacaagcgccagaaagtgtacagcctgttttatcggctggacatcgtgcctatcaatgagaaccagggctccgagtaccgcctgatcaattgcaacacctctgccatcacacaggcctgtcccaaggtgagcttcgagcctatcccaatccactattgcacccctgccggcttcgccatcctgaagtgtaaggatgagggctttaatggcacaggcctgtgcaagaacgtgtccaccgtgcagtgtacacacggcatcaagccagtggtgtctacccagctgctgctgaatggcagcctggccgagaagaacatcaccatccggtctgagaatatcacaaacaatgccaagatcatcatcgtgcagctggtgcagcctgtgaccatcaagtgcatcagaccaaacaataacacaaggaagtctatcaggatcggaccaggacaggccttttacgcaaccggcgacatcatcggcgacatcaggcaggcccactgtaacgtgacccggagcagatggaataagacactgcaggaggtggccgagaagctgcgcacctatttcggcaacaagacaatcatctttgccaatagctccggcggcgacctggagatcaccacacacagcttcaactgcggcggcgagttcttttactgtaatacctccggcctgtttaactctacctggtatgtgaacagcacatggaacgacaccgatagcacacaggagtccaacgataccatcacactgccatgcaggatcaagcagatcatcaatatgtggcagagggcaggacaggcaatgtacgcaccacctatccccggcgtgatcaagtgtgagagcaacatcaccggactgctgctgacacgggacggaggcaaggataataacgtgaacgagaccttcagaccaggaggaggcgacatgagggataattggagatccgagctgtacaagtataaggtggtggagatcgagccactgggagtggcacctacaaggtgcaagaggagagtggtggagaggcgccggagaaggcgcgcagtgggcatcggagccgtgttcctgggctttctgggagcagcaggcagcaccatgggagcagcctccatgaccctgacagtgcaggcaaggaacctgctgtccggaatcgtgcagcagcagaataacctgctgagagcaccagagtgccagcagcacctgctgaagctgaccgtgtggggcatcaagcagctgcaggccagggtgctggccgtggagcggtatctgagagaccagcagctgctgggcatctggggctgctctggcaagctgatctgctgtaccaatgtgccttggaactctagctggtccaacaagtctctggacgagatctggaataacatgacctggctgcagtgggataaggagatcaataactacacacagctgatctatcgcctgatcgaggagagccagaatcagcaggagaagaacgagaaggagctgctggagctggattgataa

SEQ ID NO: 66 - protein, 646 aa, synthetic construct
MDWTWILFLVAAATRVHSAVENLWVTVYYGVPVWRDADTTLFCASDAKAYETKKHNVWATHCCVPTDPNPQEIHLDNVTEKFNMWKNNMVEQMHTDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNITIRSENITNNAKIIIVQLVQPVTIKCIRPNNNTRKSIRIGPGQAFYATGDIIGDIRQAHCNVTRSRWNKTLQEVAEKLRTYFGNKTIIFANSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGGDMRDNWRSELYKYKVVEIEPLGVAPTRCKRRVVERRRRRRAVGIGAVFLGFLGAAGSTMGAASMTLTVQARNLLSGIVQQQNNLLRAPECQQHLLKLTVWGIKQLQARVLAVERYLRDQQLLGIWGCSGKLICCTNVPWNSSWSNKSLDEIWNNMTWLQWDKEINNYTQLIYRLIEESQNQQEKNEKELLELD

SEQ ID NO: 67 - DNA, 54 bp, synthetic construct
  source 1..54: mol_type = other DNA
atggactggacatggattctgttcctggtcgctgccgctacaagagtgcattcc

SEQ ID NO: 68 - protein, 18 aa, synthetic construct
MDWTWILFLVAAATRVHS

SEQ ID NO: 69 - DNA, 63 bp, synthetic construct
  source 1..63: mol_type = other DNA
atggagacagatacacttctgttatgggtgctgttgctgtgggtgccggggtccacaggcgac

SEQ ID NO: 70 - protein, 21 aa, synthetic construct
METDTLLLWVLLLWVPGSTGD

SEQ ID NO: 71 - DNA, 498 bp, synthetic construct
  source 1..498: mol_type = other DNA
ctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagc

SEQ ID NO: 72 - protein, 166 aa, synthetic construct
LSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS